SEQUENCE LISTING (36 sequences)

SEQ ID NO: 1 - protein, 119 aa, Rattus norvegicus
EVQLVESGGGLVQPGRSLKLSCAASGFTFSDNYLAWVRQAPEKGLEWVASISFEGISTYYGDSVKGRFTISKDNARRTLYLQMNSLRSEDTATYYCARRSYGSRAMDAWGQGTSVTVSS

SEQ ID NO: 2 - protein, 112 aa, Rattus norvegicus
DVVMTQTPVSLPVGLGGQASISCRSSQSLVHSNGNTYLSWYLKRPGQSPQLLIYRVSSRFSGVPDRFSGSGSGTDFTLKISKVVPGDLGHYYCLQTTQYPYTFGAGTKLELK

SEQ ID NO: 3 - protein, 120 aa, Mus musculus
QVQLKQSGPGLVRPSQSLSITCTVSGFSLTRFGIHWVRQSPGKGLEWLGVIWGGGSTDYDAGFISRLTITKDHSKSQVLFKINSLEADDTAMYYCATKDDDYLEAWFAHWGQGTLVTVSA

SEQ ID NO: 4 - protein, 107 aa, Mus musculus
NIVMTQSPKSVSMSVGERVTLSCKASENVGTYVSWYQQKSERSPRLLIYGASSRYTGVPDRFTGSGSATDFTLTISSVQAEDLADYHCGQSYSYPYTFGGGTKLEIK

SEQ ID NO: 5 - protein, 124 aa, Mus musculus
QVQLQQPGTELVMPGASVTLSCKASGYSFISYWMHWVKQRPGQGLEWIGEIDPSDDYTNYNQNFKGKATLTVDRSSSTAYMQLNSLTSEDSAVYYCAREENGIYYYNKGYLDVWGTGTTVTVSS

SEQ ID NO: 6 - protein, 112 aa, Mus musculus
DIVMTQTAFSNPVTLGTSASISCRSSKSLLHSNGITYLYWYLQKPGQSPQVLIYQMSNLASGVPDRFSCSGSGTDFTLRITRVEAEDVGFYYCAQNLELPYTFGGGTKLEMK

SEQ ID NO: 7 - protein, 121 aa, Mus musculus
QVQLQQPGTELVKPGASVKLSCKASGYTFTSFWMHWVKQRPGQGLEWIGNINPRNGATKYNEKFRSKVTLTADKTSSTAYMHLSSLTSEDSAVYYCAREDYDEIYYAMDSWGQGTSVTVSS

SEQ ID NO: 8 - protein, 106 aa, Mus musculus
QNVLTQSPAIMSASPGEKVTMTCSASSSVIYMYWFQQKPGSSPRVLIYDTSNLASGVPARFSGSGSGTSYSLTISRMEAEDAATYYCQQWSTYPLTFGAGTKLELK

SEQ ID NO: 9 - protein, 121 aa, Artificial Sequence
  humanized heavy variable region VH.1A
EVQLVQSGAEVKKPGASVKVSCKASGYTFTSFWMHWVRQAPGQGLEWMGNINPRNGATKYNEKFRSRVTMTADTSISTAYMELSRLRSDDTAVYYCAREDYDEIYYAMDSWGQGTTVTVSS

SEQ ID NO: 10 - protein, 121 aa, Artificial Sequence
  humanized variable heavy region VH.1B
EVQLVQSGAEVKKPGASVKVSCKASGYTFTSFWMHWVRQAPGQGLEWMGNINPRNGATKYNEKFRSRVTMTADKSISTAYMELSRLRSDDTAVYYCAREDYDEIYYAMDSWGQGTTVTVSS

SEQ ID NO: 11 - protein, 121 aa, Artificial Sequence
  humanized variable heavy region VH.1C
EVQLVQSGAEVKKPGASVKVSCKASGYTFTSFWMHWVRQAPGQGLEWMGNINPRNGATKYNEKFRSRVTLTADKSISTAYMELSRLRSDDTAVYYCAREDYDEIYYAMDSWGQGTTVTVSS

SEQ ID NO: 12 - protein, 121 aa, Artificial Sequence
  humanized variable heavy region VH.1D
EVQLVQSGAEVKKPGASVKVSCKASGYTFTSFWMHWVRQAPGQGLEWIGNINPRNGATKYNEKFRSRVTLTADKSISTAYMELSRLRSDDTAVYYCAREDYDEIYYAMDSWGQGTTVTVSS

SEQ ID NO: 13 - protein, 121 aa, Artificial Sequence
  humanized variable heavy region VH.1E
EVQLVQSGAEVKKPGASVKVSCKASGYTFTSFWMHWVRQAPGQGLEWIGNINPRNGATKYNEKFRSKVTLTADKSISTAYMELSRLRSDDTAVYYCAREDYDEIYYAMDSWGQGTTVTVSS

SEQ ID NO: 14 - protein, 106 aa, Artificial Sequence
  humanized variable light region VK.1A
ENVLTQSPATLSLSPGERATLSCSASSSVIYMYWYQQKPGQAPRLLIYDTSNLASGIPARFSGSGSGTDYTLTISSLEPEDFAVYYCQQWSTYPLTFGQGTKLEIK

SEQ ID NO: 15 - protein, 106 aa, Artificial Sequence
  humanized variable light region VK.1B
ENVLTQSPATLSLSPGERATLSCSASSSVIYMYWFQQKPGQAPRLLIYDTSNLASGIPARFSGSGSGTDYTLTISSLEPEDFAVYYCQQWSTYPLTFGQGTKLEIK

SEQ ID NO: 16 - protein, 106 aa, Artificial Sequence
  humanized variable light region VK.1C
ENVLTQSPATLSLSPGERATLSCSASSSVIYMYWFQQKPGQSPRVLIYDTSNLASGIPARFSGSGSGTDYTLTISSLEPEDFAVYYCQQWSTYPLTFGQGTKLEIK

SEQ ID NO: 17 - protein, 106 aa, Artificial Sequence
  humanized variable light region VK.1D
ENVLTQSPATLSLSPGERATLSCSASSSVIYMYWFQQKPGQSPRVLIYDTSNLASGVPARFSGSGSGTDYTLTISSLEPEDFAVYYCQQWSTYPLTFGQGTKLEIK

SEQ ID NO: 18 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 107 aa, Homo sapiens [sequence omitted]

SEQ ID NO: 20 - protein, 5 aa, Artificial Sequence
  CDR-H1 of anti-CD39 CDR Set 5
SFWMH

SEQ ID NO: 21 - protein, 17 aa, Artificial Sequence
  CDR-H2 of anti-CD39 CDR Set 5
NINPRQGATKYNEKFRS

SEQ ID NO: 22 - protein, 12 aa, Artificial Sequence
  CDR-H3 of anti-CD39 CDR Set 5
EDYDEIYYAMDS

SEQ ID NO: 23 - protein, 10 aa, Artificial Sequence
  CDR-L1 of anti-CD39 CDR Set 5
SASSSVIYMY

SEQ ID NO: 24 - protein, 7 aa, Artificial Sequence
  CDR-L2 of anti-CD39 CDR Set 5
DTSNLAS

SEQ ID NO: 25 - protein, 9 aa, Artificial Sequence
  CDR-L3 of anti-CD39 CDR Set 5
QQWSTYPLT

SEQ ID NO: 26 - protein, 5 aa, Artificial Sequence
  CDR-H1 of anti-CD39 CDR Set 6
SFWMH

SEQ ID NO: 27 - protein, 17 aa, Artificial Sequence
  CDR-H2 of anti-CD39 CDR Set 6
NINPRNAATKYNEKFRS

SEQ ID NO: 28 - protein, 12 aa, Artificial Sequence
  CDR-H3 of anti-CD39 CDR Set 6
EDYDEIYYAMDS

SEQ ID NO: 29 - protein, 10 aa, Artificial Sequence
  CDR-L1 of anti-CD39 CDR Set 6
SASSSVIYMY

SEQ ID NO: 30 - protein, 7 aa, Artificial Sequence
  CDR-L2 of anti-CD39 CDR Set 6
DTSNLAS

SEQ ID NO: 31 - protein, 9 aa, Artificial Sequence
  CDR-L3 of anti-CD39 CDR Set 6
QQWSTYPLT

SEQ ID NO: 32 - protein, 121 aa, Artificial Sequence
  variable heavy region VH.1B with G55A substitution in CDR-H2
EVQLVQSGAEVKKPGASVKVSCKASGYTFTSFWMHWVRQAPGQGLEWMGNINPRNAATKYNEKFRSRVTMTADKSISTAYMELSRLRSDDTAVYYCAREDYDEIYYAMDSWGQGTTVTVSS

SEQ ID NO: 33 - protein, 121 aa, Artificial Sequence
  variable heavy region VH.1C with G55A substitution in CDR-H2
EVQLVQSGAEVKKPGASVKVSCKASGYTFTSFWMHWVRQAPGQGLEWMGNINPRNAATKYNEKFRSRVTLTADKSISTAYMELSRLRSDDTAVYYCAREDYDEIYYAMDSWGQGTTVTVSS

SEQ ID NO: 34 - protein, 121 aa, Artificial Sequence
  variable heavy region VH.1D with G55A substitution in CDR-H2
EVQLVQSGAEVKKPGASVKVSCKASGYTFTSFWMHWVRQAPGQGLEWIGNINPRNAATKYNEKFRSRVTLTADKSISTAYMELSRLRSDDTAVYYCAREDYDEIYYAMDSWGQGTTVTVSS

SEQ ID NO: 35 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 36 - protein, 440 aa, Mus musculus
TQNKPLPENVKYGIVLDAGSSHTNLYIYKWPAEKENDTGVVQQLEECQVKGPGISKYAQKTDEIGAYLAECMELSTELIPTSKHHQTPVYLGATAGMRLLRMESEQSADEVLAAVSTSLKGYPFDFQGAKIITGQEEGAYGWITINYLLGRFTQEQSWLSLISDSQKQETFGALDLGGASTQITFVPQNSTIESPENSLQFRLYGEDYTVYTHSFLCYGKDQALWQKLAKDIQVSSGGVLKDPCFNPGYEKVVNVSELYGTPCTERFEKKLPFDQFRIQGTGDYEQCHQSILELFNNSHCPYSQCAFNGVFLPPLHGSFGAFSAFYFVMDFFKKVAKNSVISQEKMTEITKNFCSKSWEETKTSYPSVKEKYLSEYCFSGAYILSLLQGYNFTDSSWEQIHFMGKIKDSNAGWTLGYMLNLTNMIPAEQPLSPPLPHSTY

(19) United States
(12) Patent Application Publication
(10) Pub. No.: US 2022/0389111 A1
(43) Pub. Date: Dec. 8, 2022

(54) HIGH AFFINITY ANTIBODIES TO CD39 AND USES THEREOF

(71) Applicant: SHANGHAI EPIMAB BIOTHERAPEUTICS CO., LTD., Shanghai (CN)

(72) Inventors: Fang REN, Shanghai (CN); Shiyong GONG, Shanghai (CN); Chengbin WU, Shanghai (CN)

(21) Appl. No.: 17/762,128

(22) PCT Filed: Oct. 11, 2019
(86) PCT No.: PCT/CN2019/110593

§ 371 Date: Mar. 21, 2022

(30) Foreign Application Priority Data
Sep. 25, 2019 (CN) 201910907917.2

(51) Int. Cl. C07K 16/28

(52) U.S. Cl. CPC C07K 16/2896; C07K 2317/565; C07K 2317/76; C07K 2317/92; C07K 2317/24; C07K 2317/73

(57) ABSTRACT

High-affinity antibodies recognizing CD39 are disclosed. The antibodies are capable of neutralizing ATPase activity of CD39 on CD-39 expressing cells. Such antibodies are useful for treatment of cancers and other disorders mediated by CD39 activity.

### FIELD OF THE INVENTION

The present invention relates to new antibodies recognizing CD39, otherwise known as ectonucleoside triphosphate diphosphohydrolase-1 or NTPDase 1. The CD39 antibodies are useful for treatment of immunological diseases and cancers.

### BACKGROUND OF THE INVENTION

One hallmark of cancer cells is their capacity to evade immune-mediated destruction via the acquired expression of multiple negative regulators of the immune response. Such negative regulators, often referred to now as “immune checkpoints”, include surface receptors such as CTLA-4 (CD152) and PD-L1 (CD274). Targeting such key regulators of the immune response has emerged as a new treatment option to prevent tumor-mediated immunosuppression and to establish a long-lasting cancer-specific immune response. Bonnefoy et al., OncoImmunology, 4:5, e1003015 (2015). Antibody-mediated blockade of these immunoregulatory pathways has led to promising clinical results. However, because an objective response is observed in less than 50% of patients and is tumor-dependent, the identification of alternative non-redundant inhibitory/immunosuppressive pathways that could be targeted in synergistic therapeutic association schemes is of major interest.

Among the molecules involved in the regulation of the immune response, CD39 (ectonucleotidase triphosphate diphospho-hydrolase-1, NTPDase1) represents a new promising target for cancer immunotherapy. CD39 is an integral membrane protein with two transmembrane domains and a large extracellular region (Maliszewski et al, 1994) with nucleoside triphosphate diphosphohydrolase activity (Wang and Guidotti, 1996). CD39 becomes catalytically active upon its localization on the cell surface, and its glycosylation is crucial for correct protein folding, membrane targeting, and enzyme activity. (Smith et al., Biochim. Biophys. Acta., 1386: 65-78 (1998)). CD39 is constitutively expressed in spleen, thymus, lung, and placenta (Enjyoji et al., Nat. Med., 5:1010-1017 (1999); Zimmermann H., Trends Pharmacol. Sci., 20:231-236 (1999); Mizumoto et al., Nat. Med., 8:358-365 (2002); Kapojos et al., Eur. J. Pharmacol., 501:191-198 (2004)), and in these organs it is associated primarily with endothelial cells and immune cell populations, such as B cells, natural killer (NK) cells, dendritic cells, Langerhans cells, monocytes, macrophages, mesangial cells, neutrophils, and regulatory T cells (Tregs). (Dwyer et al., Purinergic Signal, 3:171-180 (2007)). CD39 expression is regulated by several pro-inflammatory cytokines, oxidative stress and hypoxia (Deaglio S. and Robson S. C., Adv. Pharmacol., 61:301-332 (2011); Eltzschig et al., Blood, 113: 224-232 (2009)), through the transcription factors Sp1 (Eltzschig et al. (2009) supra), Stat3, and zinc finger protein growth factor independent-1 transcription factor (Chalmin et al., Immunity, 36:362-373 (2012)). In addition, the expression of CD39 is increased in several solid tumors, e.g., colorectal cancer, head and neck cancer, and pancreatic cancer, as well as in chronic lymphocytic leukemia, suggesting this enzyme is also involved in the development and progression of malignancies (Bastid et al., Oncogene, 32(24):1743-1751 (2013)). A soluble catalytically active form of CD39 has been shown to circulate in human and murine blood (Yegutkin et al., FASEB. J., 26:3875-3883 (2012)).

CD39 works with CD73 to modulate the duration, magnitude and chemical nature of purinergic signals by hydrolyzing extracellular adenosine triphosphate (ATP) and adenosine diphosphate (ADP) into adenosine monophosphate (AMP) (by CD39) and AMP to adenosine (by CD73). Both CD39 and CD73 are highly expressed on regulatory T cells (Tregs, formerly referred to as suppressor T cells), which are a CD4+ subpopulation that help maintain immune system homeostasis. In cancers infiltrated with CD39-positive Tregs, CD39 plays a key role because it increases tumor angiogenesis and suppresses the immune antitumor response by initiating the generation of adenosine. (Stagg et al., Proc. Natd. Acad. Sci USA, 107(4):1547-1552 (2010)).

Myeloid-derived suppressor cells (MDSCs) also promote tumor growth by a CD39-mediated mechanism. For example, CD39 expression is elevated on MDSCs isolated from cancer patients, and these cells display inhibitory effects against antitumor T cells, as compared with MDSCs from healthy donors.

In addition, Tregs from CD39 knockout mice are constitutively activated, proliferate excessively, and have lost their suppressive function. Melanoma growth, as well as lung metastases, colon metastases, and sarcomas are also markedly decreased in knockout mice as compared to wild-type mice, with severe defects in angiogenesis also observed.

CD39 inhibition is a promising approach to overcoming Treg suppression of natural antitumor effector T cell activity. Administration of CD39 inhibitors, such as anti-CD39 antibodies and/or antigen binding fragments thereof may provide a means of restoring or supporting the antitumor immune response that is suppressed in many cancers.

In view of the progress being made in developing new approaches to the treatment of cancer by recruiting immune effector cell responses and inhibiting suppression of effector cell activation, new active CD39 inhibitor agents and methods are needed. There is also a continuing need for development of therapies directed against CD39 and evaluation of the activity of anti-CD39 antibodies, either alone or in combination with other immune checkpoint inhibitors.

### SUMMARY OF THE INVENTION

The present invention provides new antibodies that bind to CD39 with high affinity. Anti-CD39 antibodies of the present invention are useful to detect human CD39, to inhibit CD39 NTPDase1 activity, and/or to neutralize human CD39-mediated immune suppression, either in vitro or in vivo.

The invention also provides methods of making and using the anti-CD39 antibodies and/or antigen binding fragments thereof described herein as well as various compositions that may be used in methods of detecting CD39 in a sample or in methods of treating or preventing a disorder in an individual that is associated with CD39 activity.

The invention also provides novel antibodies capable of binding human CD39, wherein the antigen-binding domain of the antibody comprises a set of six CDRs, i.e., CDR-H1, CDR-H2, CDR-H3, CDR-L1, CDR-L2, and CDR-L3, selected from the group of CDR sets defined below:

| CDR | | CDR | |
|---|---|---|---|
| Set No. | CDR | Amino Acid Sequence | SEQ ID NO: |
| 1 | CDR-H1 | DNYLA | residues 31-35 of SEQ ID NO: 1 |
| | CDR-H2 | ISFEGISTYYGDSVK | residues 51-65 of SEQ ID NO: 1 |
| | CDR-H3 | RSYGSRAMDA | residues 99-108 of SEQ ID NO: 1 |
| | CDR-L1 | RSSQSLVHSNGNTYLS | residues 24-39 of SEQ ID NO: 2 |
| | CDR-L2 | VSSRF | residues 56-70 of SEQ ID NO: 2 |
| | CDR-L3 | LQTTQYPY | residues 94-101 of SEQ ID NO: 2 |
| 2 | CDR-H1 | RFGIH | residues 31-35 of SEQ ID NO: 3 |
| | CDR-H2 | VIWGGGSTDYDAGFIS | residues 50-65 of SEQ ID NO: 3 |
| | CDR-H3 | KDDDYLEAWFAH | residues 98-109 of SEQ ID NO: 3 |
| | CDR-L1 | KASENVGTYVS | residues 24-34 of SEQ ID NO: 4 |
| | CDR-L2 | GASSRYT | residues 50-56 of SEQ ID NO: 4 |
| | CDR-L3 | GQSYSYPYT | residues 89-97 of SEQ ID NO: 4 |
| 3 | CDR-H1 | SYWMH | residues 31-35 of SEQ ID NO: 5 |
| | CDR-H2 | EIDPSDDYTNYNQNFKG | residues 50-66 of SEQ ID NO: 5 |
| | CDR-H3 | EENGIYYYNKGYLDV | residues 99-113 of SEQ ID NO: 5 |
| | CDR-L1 | SSKSLLHSNGITYLY | residues 25-39 of SEQ ID NO: 6 |
| | CDR-L2 | QMSNLAS | residues 55-61 of SEQ ID NO: 6 |
| | CDR-L3 | AQNLELPYT | residues 94-102 of SEQ ID NO: 6 |
| 4 | CDR-H1 | SFWMH | residues 31-35 of SEQ ID NO: 7 |
| | CDR-H2 | NINPRNGATKYNEKFRS | residues 50-66 of SEQ ID NO: 7 |
| | CDR-H3 | EDYDEIYYAMDS | residues 99-110 of SEQ ID NO: 7 |
| | CDR-L1 | SASSSVIYMY | residues 24-33 of SEQ ID NO: 8 |
| | CDR-L2 | DTSNLAS | residues 49-55 of SEQ ID NO: 8 |
| | CDR-L3 | QQWSTYPLT | residues 88-96 of SEQ ID NO: 8 |
| 5 | CDR-H1 | SFWMH | SEQ ID NO: 20 |
| | CDR-H2 | NINPRQGATKYNEKFRS | SEQ ID NO: 21 |
| | CDR-H3 | EDYDEIYYAMDS | SEQ ID NO: 22 |
| | CDR-L1 | SASSSVIYMY | SEQ ID NO: 23 |
| | CDR-L2 | DTSNLAS | SEQ ID NO: 24 |
| | CDR-L3 | QQWSTYPLT | SEQ ID NO: 25 |
| 6 | CDR-H1 | SFWMH | SEQ ID NO: 26 |
| | CDR-H2 | NINPRNAATKYNEKFRS | SEQ ID NO: 27 |
| | CDR-H3 | EDYDEIYYAMDS | SEQ ID NO: 28 |
| | CDR-L1 | SASSSVIYMY | SEQ ID NO: 29 |
| | CDR-L2 | DTSNLAS | SEQ ID NO: 30 |
| | CDR-L3 | QQWSTYPLT | SEQ ID NO: 31 |

In an embodiment, an anti-CD39 antibody according to the invention comprises VH and VL domains, wherein the two variable domains comprise amino acid sequences selected from the group consisting of:

| | |
|---|---|
| SEQ ID NO: 1 and SEQ ID NO: 2 | SEQ ID NO: 11 and SEQ ID NO: 14 |
| SEQ ID NO: 3 and SEQ ID NO: 4 | SEQ ID NO: 11 and SEQ ID NO: 15 |
| SEQ ID NO: 5 and SEQ ID NO: 6 | SEQ ID NO: 11 and SEQ ID NO: 16 |
| SEQ ID NO: 7 and SEQ ID NO: 8 | SEQ ID NO: 11 and SEQ ID NO: 17 |
| SEQ ID NO: 9 and SEQ ID NO: 14 | SEQ ID NO: 12 and SEQ ID NO: 14 |
| SEQ ID NO: 9 and SEQ ID NO: 15 | SEQ ID NO: 12 and SEQ ID NO: 15 |
| SEQ ID NO: 9 and SEQ ID NO: 16 | SEQ ID NO: 12 and SEQ ID NO: 16 |
| SEQ ID NO: 9 and SEQ ID NO: 17 | SEQ ID NO: 12 and SEQ ID NO: 17 |
| SEQ ID NO: 10 and SEQ ID NO: 14 | SEQ ID NO: 13 and SEQ ID NO: 14 |
| SEQ ID NO: 10 and SEQ ID NO: 15 | SEQ ID NO: 13 and SEQ ID NO: 15 |
| SEQ ID NO: 10 and SEQ ID NO: 16 | SEQ ID NO: 13 and SEQ ID NO: 16 |
| SEQ ID NO: 10 and SEQ ID NO: 17 | SEQ ID NO: 13 and SEQ ID NO: 17 |
| SEQ ID NO: 32 and SEQ ID NO: 17 | SEQ ID NO: 33 and SEQ ID NO: 17 |
| SEQ ID NO: 34 and SEQ ID NO: 17. | |

In another embodiment, an anti-CD39 antibody as described herein may be used to make derivative binding proteins recognizing the same target antigen by techniques well established in the field. Such a derivative may be, e.g., a single-chain antibody (scFv), a Fab fragment (Fab), an Fab′ fragment, an F(ab′)₂, an Fv, and a disulfide linked Fv.

In another aspect of the invention, an anti-CD39 antibody described herein is capable of modulating a biological function of CD39. In another aspect, an anti-CD39 antibody described herein is capable of inhibiting CD39-mediated hydrolysis of ATP. In further embodiments, anti-CD39 antibodies according to the invention inhibit at least 80% of CD39-mediated hydrolysis of ATP, as measured in a cell-based CD39 ATPase inhibition assay.

In an embodiment, an anti-CD39 antibody described herein or an antigen-binding fragment thereof has an on rate constant (kₒₙ) to human CD39 of at least 1×10⁵ M⁻¹s⁻¹, at least 1.25×10⁵ M⁻¹s⁻¹, at least 1.35×10⁵ M⁻¹s⁻¹, at least 1.4×10⁵ M⁻¹s⁻¹, at least 1.5×10⁵ M⁻¹s⁻¹, at least 1.75×10⁵ M⁻¹s⁻¹ at least 2×10⁵ M⁻¹s⁻¹ at least 3×10⁵ M⁻¹s⁻¹ at least 5×10⁵ M⁻¹s⁻¹, at least 7×10⁵ M⁻¹s⁻¹, or at least 1×10⁶ M⁻¹s⁻¹, as measured by surface plasmon resonance or biolayer interferometry.

In another embodiment, an anti-CD39 antibody described herein or antigen-binding fragment thereof has an off rate constant (k_{off}) to human CD39 of less than 1×10⁻³s⁻¹, less than 8×10⁻⁴s⁻¹, less than 7×10⁻⁴s⁻¹, less than 6×10⁻⁴s⁻¹, less than 5×10⁻⁴s⁻¹, less than 4×10⁻⁴s⁻¹, less than 3×10⁻⁴s⁻¹, less than 1×10⁻⁴s⁻¹, less than 5×10⁻⁵s⁻¹, or less than 1×10⁻⁵s⁻¹, as measured by surface plasmon resonance or biolayer interferometry.

In another embodiment, an anti-CD39 antibody described herein or antigen-binding fragment thereof has a dissociation constant (K_{D}) to CD39 of less than 5×10⁻⁸ M, less than 1×10⁻⁸ M, less than 5×10⁻⁹ M, less than 2×10⁻⁹ M, less than 1×10⁻⁹ M, less than 8×10⁻¹⁰ M; less than 6×10⁻¹⁰ M; less than 4×10⁻¹⁰ M, less than 3×10⁻¹⁰ M, less than 2×10⁻¹⁰ M; less than 1×10⁻¹⁰ M, less than 8×10⁻¹¹ M, less than 6×10⁻¹¹ M, less than 4×10⁻¹¹ M, less than 2×10⁻¹¹ M, or less than 1×10⁻¹¹ M.

The invention also provides pharmaceutical compositions comprising at least one anti-CD39 antibody or antigen-binding fragments thereof and a pharmaceutically acceptable carrier. Pharmaceutical compositions of the invention may further comprise at least one additional active ingredient. In an embodiment, such an additional ingredient includes, but is not limited to, a therapeutic agent, an imaging agent, a cytotoxic agent, an angiogenesis inhibitor, a kinase inhibitor, a co-stimulation molecule blocker, an adhesion molecule blocker, an antibody of different specificity or functional fragment thereof, a detectable label or reporter; an agonist or antagonist for particular cytokine(s), a narcotic, a non-steroid anti-inflammatory drug (NSAID), an analgesic, an anesthetic, a sedative, a local anesthetic, a neuromuscular blocker, an antimicrobial agent, a corticosteroid, an anabolic steroid, an erythropoietin, an immunogen, an immunosuppressive agent, a growth hormone, a hormone replacement drug, a radiopharmaceutical, an antidepressant, an antipsychotic, a stimulant, a beta agonist, an inhaled steroid, an epinephrine or analog, a cytokine.

In another embodiment, a pharmaceutical composition further comprises at least one additional therapeutic agent for treating a disorder in which CD39-mediated signaling activity is detrimental.

In a further embodiment, the invention provides isolated nucleic acids encoding one or more amino acid sequences of an anti-CD39 antibody of the invention or an antigen-binding fragment thereof. Such nucleic acids may be inserted into a vector for carrying out various genetic analyses or for expressing, characterizing, or improving one or more properties of an antibody or antigen-binding fragment thereof as described herein. A vector may comprise a one or more nucleic acid molecule encoding one or more amino acid sequences of an antibody or antigen-binding fragment described herein in which the one or more nucleic acid molecule is operably linked to appropriate transcriptional and/or translational sequences that permit expression of the antibody or antigen binding fragment in a particular host cell carrying the vector. Examples of vectors for cloning or expressing nucleic acids encoding amino acid sequences of antibodies and antigen-binding fragments thereof described herein include, but are not limited, pcDNA, pTT, pTT3, pEFBOS, pBV, pJV, and pBJ.

The invention also provides a host cell comprising a vector comprising a nucleic acid encoding one or more amino acid sequences of an antibody or antigen-binding fragment thereof described herein. Host cells useful in the invention may be prokaryotic or eukaryotic. An exemplary prokaryotic host cell is Escherichia coli. Eukaryotic cells useful as host cells in the invention include protist cells, animal cells, plant cells, and fungal cells. An exemplary fungal cell is a yeast cell, including Saccharomyces cerevisiae. An exemplary animal cell useful as a host cell according to the invention includes, but is not limited to, a mammalian cell, an avian cell, and an insect cell. Preferred mammalian cells include CHO cells, HEK cells, and COS cells. An insect cell useful as a host cell according to the invention is an insect Sf9 cell.

In another aspect, the invention provides a method of producing anti-CD39 antibody or a functional fragment thereof comprising culturing a host cell comprising an expression vector encoding the antibody or functional fragment in culture medium under conditions sufficient to cause expression by the host cell of the antibody or fragment capable of binding CD39.

In one embodiment, the present invention provides methods for treating cancer in a subject in need thereof, the method comprising administering to the subject an anti-CD39 antibody or CD39-binding fragment thereof as described herein, wherein the antibody or binding fragment is capable of binding CD39 and inhibiting the ATPase activity at the surface of a cell expressing CD39.

In another embodiment, the cancer is a cancer that has not been associated with immunotherapy. In another embodiment, the cancer is a cancer that is a refractory or a recurring malignancy. In another embodiment, the anti-CD39 antibody or antigen-binding fragment thereof inhibits the growth or survival of tumor cells. In another embodiment, the cancer is selected from the group consisting of melanoma (e.g., metastatic malignant melanoma), renal cancer, pancreatic adenocarcinoma, breast cancer, colon cancer, lung cancer (e.g. non-small cell lung cancer), head and neck, liver cancer, ovarian cancer, bladder cancer, kidney cancer, salivary cancer, stomach cancer, gliomas cancer, thyroid cancer, thymic cancer, epithelial cancer, gastric cancer and lymphoma.

### BRIEF DESCRIPTION OF THE DRAWINGS

FIG. 1 are concentration plots comparing the performance of murine anti-human CD39 mAbs in a cell-based CD39 ATPase inhibition assay. Plots for anti-CD39 monoclonal antibodies mAb628, mAb629, mAb634, mAb635, mAb636, mAb638, and an irrelevant murine IgG control are presented. It can be seen that mAb638 achieves essentially complete inhibition of cell-based CD39 ATPase activity at sub-nanomolar concentration.

FIG. 2 shows concentration plots of the performance of two rat anti-mouse CD39 (muCD39) antibodies in a protein-based CD39 ATPase inhibition assay. Plots for anti-muCD39 monoclonal antibodies mAb605 and mAb606, and an irrelevant rat IgG control are presented. It can be seen that both anti-muCD39 antibodies tested achieve EC50 at concentrations of 30-50 nM.

FIG. 3 shows concentration plots comparing the performance of humanized anti-human CD39 mAbs in a cell-based CD39 ATPase inhibition assay. Plots for humanized anti-huCD39 monoclonal antibodies HuEM0004-38-21, -22, and -23, murine anti-huCD39 mAb638, an irrelevant human IgG control and an irrelevant murine IgG control are presented. It can be seen that the humanized antibodies performed similarly, achieving essentially complete inhibition of ATPase activity at concentrations near 1 nM. Murine mAb638 was also effective but was outperformed by the humanized antibodies incorporating the mAb638 CDR set (with a G55A mutation in CDR-H2, see Example 2.3, infra).

FIG. 4 is a series of bar graphs showing the effect of increasing anti-CD39 activity in ATPase-mediated T cell suppression in a T cell proliferation assay. At 100 nM concentrations, ATPase activity is neutralized by the murine anti-huCD39 antibody mAb638, leading to restored proliferation of activated T cells in the presence of ATP.

FIG. 5, FIG. 6, and FIG. 7 each present a series of bar graphs showing the effect on ATPase neutralization in the T cell proliferation suppression assay with increasing concentration of humanized anti-huCD39 antibody added to the activated T cell/ATP mixtures. Bars showing the neutralization effect at varying concentrations of HuEM0004-38-21 (FIG. 5), HuEM0004-38-22 (FIG. 6), and HuEM0004-38-23 (FIG. 7) are presented. These data show that the humanized antibodies retain the ATPase neutralization properties of the murine antibody having the same CDR set (except for the point mutation G54A to eliminate an NG site in CDR-H2, see Example 2.3).

### DETAILED DESCRIPTION OF THE INVENTION

This invention pertains to novel anti-CD39 antibodies and antigen-binding portions thereof useful to detect human CD39, to inhibit CD39 NTPDase1 activity, and/or to neutralize human CD39-mediated immune suppression, either in vitro or in vivo.

The invention also provides methods of making and using the anti-CD39 antibodies and/or antigen binding fragments thereof described herein as well as various compositions that may be used in methods of detecting CD39 in a sample or in methods of treating or preventing a disorder in an individual that is associated with CD39 activity.

Various aspects of the invention relate to anti-CD39 antibodies and antibody fragments, and pharmaceutical compositions thereof, as well as nucleic acids, recombinant expression vectors and host cells for making such antibodies and functional antibody fragments. Methods of using the antibodies and functional antibody fragments of the invention to detect human CD39 to inhibit human CD39 activity, either in vitro or in vivo; and to treat diseases, especially cancer, that are mediated by CD39-mediated ATPase activity are also encompassed by the invention.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the present invention may be more readily understood, select terms are defined below.

The term “human CD39” (abbreviated herein as huCD39) is intended to include recombinant human CD39 which can be prepared by standard recombinant expression methods. The polypeptide sequence of human CD39 is shown in the table below (extracellular domain (ECD) underlined):

Amino Acid Sequence of Human CD39
12345678901234567890123456789012345678901234567890

MEDTKESNVKTFCSKNILAILGFSSIIAVIALLAVGLTQNKALPENVKYG
IVLDAGSSHTSLYIYKWPAEKENDTGVVHQVEECRVKGPGISKFVQKVNE
IGIYLTDCMERAREVIPRSQHQETPVYLGATAGMRLLRMESEELADRVLD
VVERSLSNYPFDFQGARIITGQEEGAYGWITINYLLGKFSQKTRWFSIVP
YETNNQETFGALDLGGASTQVTFVPQNQTIESPDNALQFRLYGKDYNVYT
HSFLCYGKDQALWQKLAKDIQVASNEILRDPCFHPGYKKVVNVSDLYKTP
CTKRFEMTLPFQQFEIQGIGNYQQCHQSILELFNTSYCPYSQCAFNGIFL
PPLQGDFGAFSAFYFVMKFLNLTSEKVSQEKVTEMMKKFCAQPWEEIKTS
YAGVKEKYLSEYCFSGTYILSLLLQGYHFTADSWEHIHFIGKIQGSDAGW
TLGYMLNLTNMIPAEQPLSTPLSHSTYVFLMVLFSLVLFTVAIIGLLIFH
KPSYFWKDMV (SEQ ID NO: 35)

The term “polypeptide” refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein amino acid sequence. The term “polypeptide” encompasses fragments and variants (including fragments of variants) thereof, unless otherwise contradicted by context. For an antigenic polypeptide, a fragment of polypeptide optionally contains at least one contiguous or nonlinear epitope of polypeptide. The precise boundaries of the at least one epitope fragment can be confirmed using ordinary skill in the art. The fragment comprises at least about 5 contiguous amino acids, such as at least about 10 contiguous amino acids, at least about 15 contiguous amino acids, or at least about 20 contiguous amino acids. A variant of as polypeptide is as described herein.

The term “isolated protein” or “isolated polypeptide” is a protein or polypeptide that by virtue of its origin or source of derivation is not associated with naturally associated components that accompany it in its native state, is substantially free of other proteins from the same species, is expressed by a cell from a different species, or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” refers to the process of rendering a chemical species such as a polypeptide substantially free of naturally associated components by isolation, e.g., using protein purification techniques well known in the art.

The term “biological activity” refers to all inherent biological properties of the CD39 protein. Biological properties of CD39 include, but are not limited to, nucleoside triphosphate diphosphohydrolase activity. CD39 has the ability to modulate the duration, magnitude and chemical nature of purinergic signals by hydrolyzing extracellular adenosine triphosphate (ATP) and adenosine diphosphate (ADP) into adenosine monophosphate (AMP).

The term “specific binding” or “specifically binding” in reference to the interaction of an antibody, an antigen-binding portion thereof, or a peptide with a second chemical species, means that the interaction is dependent upon the presence of a particular structure (e.g., an antigenic determinant or epitope) on the second chemical species. For example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody” broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as VH) and a heavy chain constant region. The heavy chain constant region is comprised of three domains: CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is comprised of three CDRs and four FRs, arranged from amino-terminus to carboxy-terminus in the following order: FR1, CDR1, FR2, CDR2, FR3, CDR3, FR4. First, second and third CDRs of a VH domain are commonly enumerated as CDR-H1, CDR-H2, and CDR-H3; likewise, first, second and third CDRs of a VL domain are commonly enumerated as CDR-L1, CDR-L2, and CDR-L3. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain, and a CH3 domain, and optionally comprises a CH4 domain. Variant Fc regions having replacements of amino acid residues in the Fc portion to alter antibody effector function are known in the art (see, e.g., Winter et al., U.S. Pat. Nos. 5,648,260 and 5,624,821). The Fc portion of an antibody mediates several important effector functions, for example, cytokine induction, ADCC, phagocytosis, complement dependent cytotoxicity (CDC), and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region that connects CH1 constant domains to the Fc constant domains (e.g., CH2 and CH3). The anti-inflammatory activity of IgG is completely dependent on sialylation of the N-linked glycan of the IgG Fc fragment. The precise glycan requirements for anti-inflammatory activity has been determined, such that an appropriate IgG1 Fc fragment can be created, thereby generating a fully recombinant, sialylated IgG1 Fc with greatly enhanced potency (see, Anthony et al., Science, 320:373-376 (2008)).

The terms “antigen-binding portion” and “antigen-binding fragment” or “functional fragment” of an antibody are used interchangeably and refer to one or more fragments of an antibody that retain the ability to specifically bind to an antigen, i.e., the same antigen (e.g., CD39) as the full-length antibody from which the portion or fragment is derived. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature, 341: 544-546 (1989); PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al., Science, 242: 423-426 (1988); and Huston et al., Proc. Natl. Acad. Sci. USA, 85: 5879-5883 (1988)). Such single chain antibodies are also intended to be encompassed within the term “antigen-binding portion” of an antibody and equivalent terms given above. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. USA, 90: 6444-6448 (1993). Such antibody binding portions are known in the art (Kontermann and Dübel eds., Antibody Engineering (Springer-Verlag, New York, 2001), p. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al., Protein Eng., 8(10): 1057-1062 (1995); and U.S. Pat. No. 5,641,870)).

An immunoglobulin constant (C) domain refers to a heavy (CH) or light (CL) chain constant domain. Murine and human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic determinant (epitope). Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human antibody” includes antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell, antibodies isolated from a recombinant, combinatorial human antibody library (Hoogenboom, H. R., Trends Biotechnol., 15: 62-70 (1997); Azzazy and Highsmith, Clin. Biochem., 35: 425-445 (2002); Gavilondo and Larrick, BioTechniques, 29: 128-145 (2000); Hoogenboom and Chames, Immunol. Today, 21: 371-378 (2000)), antibodies isolated from an animal (e.g., a mouse) that is transgenic for human immunoglobulin genes (see, e.g., Taylor et al., Nucl. Acids Res., 20: 6287-6295 (1992); Kellermann and Green, Curr. Opin. Biotechnol., 13: 593-597 (2002); Little et al., Immunol. Today, 21: 364-370 (2000)); or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs has been replaced with murine CDR sequences.

The term “humanized antibody” refers to antibodies that comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which CDR sequences from a non-human species (e.g., mouse) are introduced into human VH and VL framework sequences. A humanized antibody is an antibody or a variant, derivative, analog or fragment thereof which immunospecifically binds to an antigen of interest and which comprises framework regions and constant regions having substantially the amino acid sequence of a human antibody but complementarity determining regions (CDRs) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody may be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the acceptor framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, preferably at least 85%, more preferably at least 90%, and most preferably at least 95% of the humanized antibody residues will correspond to those of the parental FR and CDR sequences. Back mutation at a particular framework position to restore the same amino acid that appears at that position in the donor antibody is often utilized to preserve a particular loop structure or to correctly orient the CDR sequences for contact with target antigen.

The term “CDR” refers to the complementarity determining regions within antibody variable domain sequences. There are three CDRs in each of the variable regions of the heavy chain and the light chain, which are designated CDR-H1, CDR-H2, CDR-H3, CDR-L1, CDR-L2, and CDR-L3. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs.

The term “Kabat numbering”, which is recognized in the art, refers to a system of numbering amino acid residues which are more variable (i.e., hypervariable) than other amino acid residues in the heavy and light chain variable regions of an antibody or an antigen-binding portion thereof. See, Kabat et al., Ann. NY Acad. Sci., 190: 382-391 (1971); and Kabat et al., Sequences of Proteins of Immunological Interest, Fifth Edition, U.S. Department of Health and Human Services, NIH Publication No. 91-3242 (1991).

The term “multivalent binding protein” denotes a binding protein comprising two or more antigen binding sites. A multivalent binding protein is preferably engineered to have three or more antigen binding sites, and is generally not a naturally occurring antibody.

The term “activity” includes properties such as the ability to bind a target antigen with specificity, the affinity of an antibody for an antigen, the ability to neutralize the biological activity of a target antigen, the ability to inhibit interaction of a target antigen with its natural receptor(s), and the like. Preferred antibodies and antigen-binding portions thereof of the present invention have the ability to inhibit the ATPase activity of CD39.

The term “kₒₙ” (also “Kon”, “kon”), as used herein, is intended to refer to the on rate constant for association of a binding protein (e.g., an antibody) to an antigen to form an association complex, e.g., antibody/antigen complex, as is known in the art. The “kₒₙ” also is known by the terms “association rate constant”, or “ka”, as used interchangeably herein. This value indicates the binding rate of an antibody to its target antigen or the rate of complex formation between an antibody and antigen as is shown by the equation below:

Antibody (“Ab”)+Antigen (“Ag”)→Ab−Ag.

The term “k_{off}” (also “Koff”, “koff”), as used herein, is intended to refer to the off rate constant for dissociation, or “dissociation rate constant”, of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab−Ag.

The term “K_{D}”, as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Biolayer interferometry using, e.g., the Octet® RED96 system (Pall FortéBio LLC), is another affinity assay technique. Additionally, a KinExA® (Kinetic Exclusion Assay) assay, available from Sapidyne Instruments (Boise, Id.) can also be used. Antibodies and antigen-binding fragments thereof of the present invention are considered to have “high affinity” for a target antigen (e.g., human CD39) if their K_{D}, as measured by surface plasmon resonance or biolayer interferometry is subnanomolar (i.e., K_{D}<10⁻⁹ M).

The term “isolated nucleic acid” shall mean a polynucleotide (e.g., of genomic, cDNA, or synthetic origin, or some combination thereof) that, by human intervention, is not associated with all or a portion of the polynucleotides with which it is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NS0, SP2 and PER.C6; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual, 2nd ed. (Cold Spring Harbor Laboratory Press, Cold Spring Harbor, N.Y., 1989).

The term “agonist”, as used herein, refers to a modulator that, when contacted with a molecule of interest, causes an increase in the magnitude of a certain activity or function of the molecule compared to the magnitude of the activity or function observed in the absence of the agonist. The terms “antagonist” and “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human CD39.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, or progression of one or more symptoms associated with a disorder; detect a disorder; or enhance or improve the prophylactic or therapeutic effect(s) of another therapy (e.g., prophylactic or therapeutic agent).

### Production of Anti-CD39 Antibodies

Anti-CD39 antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr− CHO cells, described in Urlaub and Chasin, Proc. Natl. Acad. Sci. USA, 77: 4216-4220 (1980), used with a DHFR selectable marker, e.g., as described in Kaufman and Sharp, J Mol. Biol., 159: 601-621 (1982)), NS0 myeloma cells, COS cells, HEK293 cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In an exemplary system for recombinant expression of an antibody, or antigen-binding portion thereof, of the invention, a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of making a recombinant anti-CD39 antibody of the invention by culturing a transfectected host cell of the invention in a suitable culture medium until a recombinant antibody of the invention is produced. The method can further comprise isolating the recombinant antibody from the culture medium.

### Uses of Antibodies of the Invention

Given their ability to bind to CD39, the antibodies described herein and functional fragments thereof can be used to detect CD39, e.g., in a biological sample containing cells that express CD39. The antibodies and functional fragments of the invention can be used in a conventional immunoassay, such as an enzyme linked immunosorbent assays (ELISA), a radioimmunoassay (RIA), or tissue immunohistochemistry. The invention provides a method for detecting CD39 in a biological sample comprising contacting a biological sample with an antibody or antigen-binding portion thereof of the invention and detecting whether binding to a target antigen occurs, thereby detecting the presence or absence of the target in the biological sample. The antibody or functional fragment may be directly or indirectly labeled with a detectable substance to facilitate detection of the bound or unbound antibody/fragment. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include 3H, ¹⁴C, 35, ⁹⁰Y ⁹⁹Tc, ¹¹¹In, ¹²⁵I ¹³¹I ¹⁷⁷Lu, ¹⁶⁶Ho, or ¹⁵³Sm.

The antibodies and antibody fragments of the invention preferably are capable of neutralizing human CD39 activity both in vitro and in vivo. Accordingly, they can be used to inhibit CD39 enzymatic activity (ATPase activity) and/or inhibit CD39-mediated ATP and ADP hyrolysis in a cell culture containing CD39-expressing cells, in human subjects, or in other mammalian subjects having CD39 with which an antibody or antibody fragment of the invention cross-reacts.

In another embodiment, the invention provides a method for treating a subject suffering from a disease or disorder in which CD39 activity is detrimental, such method comprising administering to the subject an antibody or antigen-binding fragment thereof of the invention such that activity mediated by CD39 activity in the cellular microenvironment in the subject is reduced.

As used herein, the term “a disorder in which CD39 activity is detrimental” is intended to include diseases and other disorders in which the ATPase activity of CD39, or the consequences of it, in a subject suffering from the disorder is either responsible for the pathophysiology of the disorder or is a factor that contributes to a worsening of the disorder. Accordingly, a disorder in which CD39 activity is detrimental is a disorder in which inhibition of CD39 activity is expected to alleviate the symptoms and/or progression of the disorder.

Anti-CD39 antibodies and antigen-binding fragments thereof as disclosed herein will be useful for treatment of diseases and disorders where inhibition of ATPase activity is desirable. Such disorders include, for example, many cancers including melanoma, lung cancer, breast cancer, ovarian cancer, gastric cancer, liver cancer, and lymphoma. Such antibodies and antigen-binding fragments may also be used for treating infectious diseases, such as HIV, hepatitis B, hepatitis C, and bacterial infection.

The invention also provides pharmaceutical compositions comprising an antibody, or antigen-binding portion thereof, and a pharmaceutically acceptable carrier. The pharmaceutical compositions comprising the antibodies and/or antigen-binding portions thereof of the invention are for use in, but not limited to, diagnosing, detecting, or monitoring a disorder, in treating, managing, or ameliorating of a disorder or one or more symptoms thereof, and/or in research. In a specific embodiment, a composition comprises one or more antibodies of the invention. In another embodiment, the pharmaceutical composition comprises one or more antibodies of the invention and one or more prophylactic or therapeutic agents other than antibodies of the invention for treating a disorder in which CD39 activity is detrimental. In an embodiment, the prophylactic or therapeutic agents are known to be useful for, or have been or are currently being used in the prevention, treatment, management, or amelioration of, a disorder or one or more symptoms thereof. In accordance with these embodiments, the composition may further comprise a carrier, diluent or excipient.

The antibodies and/or antigen-binding portions thereof of the invention can be incorporated into pharmaceutical compositions suitable for administration to a subject. Typically, the pharmaceutical composition comprises an antibody or antigen-binding portion thereof of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), intratumoral, transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic, such as lignocaine, to ease pain at the site of the injection.

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

An antibody or functional fragment thereof, of the invention also can be administered with one or more additional therapeutic agents useful in the treatment of various diseases. Antibodies and functional fragments thereof, described herein can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody or functional fragment thereof, of the present invention. The additional agent also can be an agent that imparts a beneficial attribute to the therapeutic composition, e.g., an agent that affects the viscosity of the composition.

Having now described the present invention in detail, the same will be more clearly understood by reference to the following examples, which are included for purposes of illustration only and are not intended to be limiting of the invention.

### EXAMPLES

### Example 1: Generation of Anti-CD39 Monoclonal Antibodies

Mouse anti-human CD39 and Rat anti-mouse CD39 monoclonal antibodies were obtained as follows:

### Example 1.1(a): Immunization of Mice with Human CD39 Antigen

Fifty micrograms of recombinant purified human CD39 protein (R&D Systems, Inc., Minneapolis, Minn., USA) mixed with complete Freund's adjuvant or 5×10⁶ cells of a CHO-K1-humanCD39 stable cell line without adjuvant were injected intraperitoneally into two groups of five 6-8-week old Balb/C and SJL mice on Day 1. On days 14 and 35 twenty-five micrograms of recombinant purified human CD39 protein mixed with incomplete Freund's adjuvant or 5×10⁶ of CHO-K1-humanCD39 stable cell line without adjuvant were injected intraperitoneally into the same mice. A final boost with the same immunogens was done 3-4 days before fusion.

Example 1.1(b): Immunization of Rats with Mouse CD39 Antigen One hundred micrograms of recombinant purified mouse CD39 protein (Chempartner Co., Ltd.; Shanghai) mixed with complete Freund's adjuvant were injected intraperitoneally into one 6-8-week old Sprague Dawley rat on Day 1. On days 14 and 35 fifty micrograms of recombinant purified mouse CD39 protein mixed with incomplete Freund's adjuvant were injected intraperitoneally into the same rat. A final boost with same immunogen was done 3-4 days before fusion.

### Example 1.2: Generation of Hybridomas

Splenocytes obtained from the immunized mice and rat described in Example 1.1(a) and (b) were fused with SP2/O-Ag-14 cells at a ratio of 5:1 according to the established method described in Kohler and Milstein, Nature, 256:495 (1975) to generate hybridomas. Fusion products were plated in selection media containing hypoxanthine-aminopterin-thymidine (HAT) in 96-well plates at a density of 1×10⁵ spleen cells per well. Seven to ten days post-fusion, macroscopic hybridoma colonies were observed.

Supernatant from each well containing hybridoma colonies was tested by ELISA or FACS for the presence of antibody to CD39.

### CD39 Enzyme-Linked Immunosorbent Assay (ELISA)

To determine if anti-CD39 mAbs bind to human CD39, ELISA plates were incubated overnight at 4° C. with human CD39 protein or mouse CD39 protein diluted in PBS, pH7.4 buffer at 1 μg/ml. Plates were washed four times in washing buffer (PBS containing 0.05% Tween 20), and blocked for 1 hour at 37° C. with 200 μl per well blocking buffer (1% BSA in PBS containing 0.05% Tween 20). After blocking buffer was removed, hybridoma supernatant or diluted purified Abs were added to the wells at 100 μl per well and incubated at 37° C. for 1 hour. The wells were washed four times with washing buffer, and anti-mouse HRP (for mouse anti-human CD39 Ab characterization) or anti-rat HRP (for rat anti-mouse CD39 Ab characterization) (Sigma) were 1:5000 diluted and added to the wells at 100 μl per well. The plates were incubated for 1 hour at 37° C. and washed four times in washing buffer. 100 μl of tetramethylbenzidine (TMB) chromogenic solution were added per well. Following color development, the reaction was stopped with 1N HCl, and absorbance at 450 nM was measured. The data were proceeded by GraphPad software.

### Cell Membrane CD39 Binding Assay

The ability of the purified antibodies to bind to cell membrane human CD39, cynomolgus monkey CD39, or mouse CD39 was determined by FACS analysis. CHO-K1 cells stably transfected to overexpress human CD39, cynomolgus monkey CD39, or mouse CD39 (CHO-K1-huCD39 cells, CHO-K1-cyCD39 cells, and CHOK1-muCD39 cells) were generated for use in this assay. Briefly, SK-MEL-28 melanoma cells (ATCC) or CD39 overexpressing stable CHO cell lines were resuspended in PBS containing 2% FBS (FACS buffer) and seeded at 1-5×10⁵ cells/well into U-bottomed plates. Anti-CD39 mAbs or isotype control antibodies diluted in FACS buffer were added to the wells and incubated for 1 hour at 4° C. After washing with FACS buffer, 1:1000 diluted Fluorescence-labeled secondary antibody (Life Technologies/ThermoFisher Scientific) were added and incubated for 30 min. at 4° C. Unbound secondary mAb was removed by washing with FACS buffer three times, and the samples were then read on a FACS instrument. The data were processed by GraphPad software.

### Example 1.3: Identification and Characterization of Anti-Human CD39 Antibodies

Hybridomas producing antibodies that bind human CD39 and are capable of binding CD39 specifically were scaled-up and cloned by limiting dilution.

Monoclonal hybridoma cells were expanded into hybridoma serum-free media containing 2.5% low IgG fetal bovine serum. On average, 200 mL of each hybridoma supernatant (derived from a clonal population) were harvested, concentrated, and purified by protein A affinity chromatography by standard methods. The ability of purified mAbs to bind CD39 were tested using the ELISA and FACS (cell membrane CD39 binding) assays described above. The ability of mAbs to inhibit CD39 enzyme activity was determined using the protein-based and cell-based ATPase activity assays described below.

### Inhibition of Protein-Based CD39 ATPase Activity

The ability of the purified anti-mouse or anti-human CD39 antibodies to inhibit CD39 ATPase activity was determined by protein-based analysis. Anti-mouse or anti-human CD39 antibodies were serially diluted in assay buffer (10 mM glucose, 20 mM Hepes, 5 mM KCl, 120 mM NaCl, 2 mM CaCl2), pH 7.5) and added to an assay plate (Perkin Elmer, Catalog #6005181) at 50 μl/well. Recombinant CD39 protein diluted to 0.12 μg/ml, and 25 μl/well was added to the assay plate. The assay plate was incubated at 4° C. for 30 min. before 40 μM ATP substrate was added to the plate at 25 μl/well and incubated at 37° C. for 30 min. The remaining ATP was tested by CellTiter-Glo® Luminescent Cell Viability Assay (Promega, Catalog #G7573). The data were processed using GraphPad software.

### Inhibition of Cell-Surface Human CD39 ATPase Activity

The ability of the purified anti-human CD39 antibodies to inhibit human CD39 ATPase activity was determined by cell-based analysis. SK-MEL-28 human melanoma cells, which endogenously express CD39 on the cell surface, were originally obtained from ATCC and subcultured in Eagle's Minimal Essential Medium (EMEM) with 10% FBS and grown at 37° C. in a 5% CO₂ incubator. Cells were harvested and seeded into wells of a 96-well plate at 2×10⁵ cells/ml, 50 μl/well. Anti-human CD39 antibodies were serially diluted in assay buffer (10 mM glucose, 20 mM Hepes, 5 mM KCl, 120 mM NaCl, 2 mM CaCl₂, pH 7.5) and added to the SK-MEL-28 cells at 50 μl/well. The cells and antibodies mixture was incubated at 37° C. overnight. After the cells were washed three times with assay buffer, 100 μM ATP was added and incubated at 37° C. for 25 min. Supernatant was transferred to a new 96-well plate and phosphate concentration in supernatants was measured according to the procedure of Malachite Green Phosphate Detection Kit (R&D Systems; Cat. No. DY996).

The specific binding activity of anti-human CD39 antibodies to CHO-K1-human CD39 and CHO-K1-cynomolgus CD39 stable cell lines is shown in Table 1. The binding activity of anti-mouse CD39 antibodies to mouse CD39 protein and the CHO-K1-mouse CD39 stable cell line is shown in Table 2. The data were processed using Graphpad software.

TABLE 1
Cell-Based Antibody Binding Activity
to Human/Cynomolgus CD39

| Anti-CD39 | | CHO-K1- | CHO-K1- |
|---|---|---|---|
| Antibody | | human CD39 | cyno CD39 |
| Antibody ID | Isotype | EC50(nM) | EC50(nM) |
| mAb623 | IgG2a, κ | 1.45 | |
| mAb624 | IgG2a, κ | 1.80 | |
| mAb625 | IgG2a, κ | 1.84 | |
| mAb626 | IgG2b, κ | 1.85 | |
| mAb627 | IgG2b, κ | 1.74 | |
| mAb628 | IgG2a, κ | 3.37 | |
| mAb629 | IgG2a, κ | 1.92 | 2.18 |
| mAb630 | IgG2a, κ | 195.00 | |
| mAb631 | IgG2a, κ | 1.72 | |
| mAb632 | IgG2a, κ | 1.72 | |
| mAb633 | IgG2a, κ | 2.19 | |
| mAb634 | IgG2a, κ | 1.86 | |
| mAb635 | IgG2a, κ | 3.53 | 4.79 |
| mAb636 | IgG2a, κ | 12.66 | 10.60 |
| mAb637 | IgG2b, κ | 7.94 | |
| mAb638 | IgG1, κ | 3.34 | 19.19 |
| mAb639 | IgG1, κ | 0.81 | |
| mAb640 | IgG2b, κ | 1.42 | |
| mAb641 | IgG1, κ | 6.17 | |
| mAb642 | IgG1, κ | 6.79 | |
| mAb643 | IgG2b, κ | 15.95 | |
| mAb644 | IgG2b, κ | 14.31 | |
| mAb645 | IgG1, κ | 2.50 | |
| mAb646 | IgG2b, κ | 1.92 | 1.40 |
| mAb647 | IgG2b, κ | 1.71 | 1.76 |
| mAb648 | IgG2b, κ | 2.09 | 1.81 |
| mAb649 | IgG2c, κ | 4.75 | 3.63 |
| mAb650 | IgG2c, κ | 7.53 | 3.84 |
| mAb652 | IgG2b, κ | 40.93 | 22.54 |
| mAb653 | IgG1, κ | 9.50 | 6.27 |

TABLE 2
Anti-mouse CD39 Antibody Binding Activity vs. Mouse CD39 Protein
(ELISA) and CHO-K1-mouse CD39 Stable Cell Line (FACS)

| Anti-CD39 | | | |
|---|---|---|---|
| Antibody | | ELISA | FACS |
| Antibody ID | Isotype | EC50(nM) | EC50(nM) |
| mAb601 | IgG2a, κ | 0.20 | 1.94 |
| mAb602 | IgG1 | 0.17 | 6.38 |
| mAb604 | IgG2a, κ | 0.17 | 2.95 |
| mAb605 | IgG2a, κ | 0.09 | 2.07 |
| mAb606 | IgG2a, κ | 0.17 | 1.16 |
| mAb607 | IgG1, κ | 3.01 | 6.09 |
| mAb608 | IgG1, λ | 0.35 | 1.55 |
| mAb609 | IgG2a, κ | 0.24 | 19.74 |
| mAb610 | IgG1, λ | 0.40 | 1.46 |
| mAb611 | IgG1, κ | 0.84 | 5.34 |
| mAb612 | IgG2a, κ | 0.72 | 8.73 |
| mAb613 | IgG2a, κ | 0.82 | 7.88 |
| mAb614 | IgG2a, λ | 1.71 | 19.83 |
| mAb615 | IgG2a, λ | 0.12 | 1.25 |
| mAb616 | IgG2a, κ | 0.42 | 30.86 |
| mAb617 | IgG2a, κ | 0.71 | 5.18 |
| mAb618 | IgG2a, λ | 0.17 | 6.35 |
| mAb619 | IgG2a, κ | 0.11 | 1.75 |
| mAb620 | IgG2a, κ | 0.47 | 27.07 |
| mAb621 | IgG2a, κ | 0.46 | 38.17 |
| mAb622 | IgG1, κ | 2.91 | 1.60 |

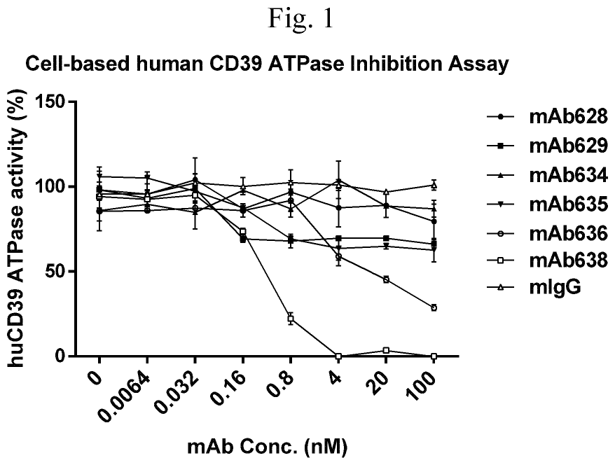

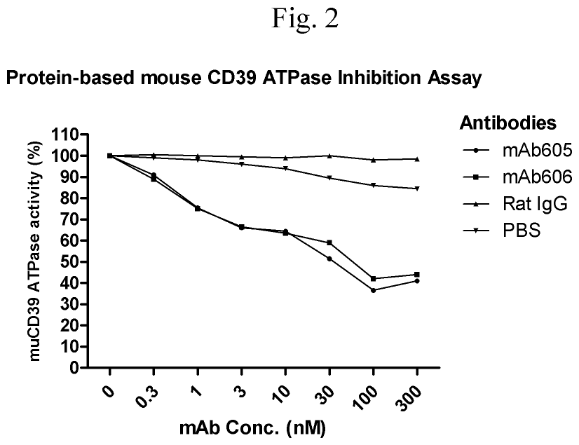

The inhibition of CD39-mediated ATPase activity by anti-human CD39 antibodies is shown in FIG. 1. The inhibition of CD39-mediated ATPase activity by anti-mouse CD39 antibodies is shown in FIG. 2.

### Example 1.4: Sequencing of Murine Anti-huCD39 Antibody Variable Regions

To amplify heavy and light chain variable regions, total RNA of each hybridoma clone was isolated from >5×10⁶ cells with TRIzol® RNA isolation reagent (Cat. No. 15596, Invitrogen). cDNA was synthesized by SuperScript™ III First-Strand Synthesis SuperMix (Cat. No. 18080, Invitrogen) and applied as a PCR template of Mouse Ig-Primer Set (Cat. No. 69831-3, Novagen). PCR amplified products were analyzed by electrophoresis on a 1.2% agarose gel with SYBR™ Safe DNA gel stain (Invitrogen). DNA fragments with correct size were purified with NucleoSpin® Gel and PCR Clean-up (#740609, Macherey-Nagel GmbH) according to manufacturer's instructions and subcloned to pMD18-T cloning vector (Sino Biological Inc.) individually. Fifteen colonies from each transformation were selected and sequences of insert fragments were analyzed by DNA sequencing. Sequences were confirmed if at least 8 match consensus sequences for VH and VL. Four mAbs were selected based on ATPase inhibition activity, and the protein sequences of the four mAbs variable regions were analyzed by sequence homology alignment and listed in Table 3. Complementarity determining regions (CDRs) in the variable domains were identified based on Kabat numbering system and appear underlined in Table 3 below.

TABLE 3
Protein Sequences of One Anti-Mouse CD39 Antibody (mAb605) and Three
Anti-Human CD39 Antibodies (mAb629, mAb636 and mAb638)

| | | SEQ ID | protein sequences |
|---|---|---|---|
| mAb ID | Domain | NO. | 1234567890123456789012345678901234567890 |
| mAb605 | VH | 1 | EVQLVESGGGLVQPGRSLKLSCAASGFTFSDNYLAWVRQA |
| | | | PEKGLEWVASISFEGISTYYGDSVKGRFTISKDNARRTLY |
| | | | LQMNSLRSEDTATYYCARRSYGSRAMDAWGQGTSVTVSS |
| | VL | 2 | DVVMTQTPVSLPVGLGGQASISCRSSQSLVHSNGNTYLSW |
| | | | YLKRPGQSPQLLIYRVSSRFSGVPDRFSGSGSGTDFTLKI |
| | | | SKVVPGDLGHYYCLQTTQYPYTFGAGTKLELK |
| mAb629 | VH | 3 | QVQLKQSGPGLVRPSQSLSITCTVSGFSLTRFGIHWVRQS |
| | | | PGKGLEWLGVIWGGGSTDYDAGFISRLTITKDHSKSQVLF |
| | | | KINSLEADDTAMYYCATKDDDYLEAWFAHWGQGTLVTVSA |
| | VL | 4 | NIVMTQSPKSVSMSVGERVTLSCKASENVGTYVSWYQQKS |
| | | | ERSPRLLIYGASSRYTGVPDRFTGSGSATDFTLTISSVQA |
| | | | EDLADYHCGQSYSYPYTFGGGTKLEIK |
| mAb636 | VH | 5 | QVQLQQPGTELVMPGASVTLSCKASGYSFISYWMHWVKQR |
| | | | PGQGLEWIGEIDPSDDYTNYNQNFKGKATLTVDRSSSTAY |
| | | | MQLNSLTSEDSAVYYCAREENGIYYYNKGYLDVWGTGTTV |
| | | | TVSS |
| | VL | 6 | DIVMTQTAFSNPVTLGTSASISCRSSKSLLHSNGITYLYW |
| | | | YLQKPGQSPQVLIYQMSNLASGVPDRFSCSGSGTDFTLRI |
| | | | TRVEAEDVGFYYCAQNLELPYTFGGGTKLEMK |
| mAb638 | VH | 7 | QVQLQQPGTELVKPGASVKLSCKASGYTFTSFWMHWVKQR |
| | | | PGQGLEWIGNINPRNGATKYNEKFRSKVTLTADKTSSTAY |
| | | | MHLSSLTSEDSAVYYCAREDYDEIYYAMDSWGQGTSVTVS |
| | | | S |
| | VL | 8 | QNVLTQSPAIMSASPGEKVTMTCSASSSVIYMYWFQQKPG |
| | | | SSPRVLIYDTSNLASGVPARFSGSGSGTSYSLTISRMEAE |
| | | | DAATYYCQQWSTYPLTFGAGTKLELK |

### Example 2: Humanization of Murine Anti-CD39 Antibodies

Based on the specificity and cell surface human CD39 binding activity, cynomolgus CD39 cross-reactivity, and ATPase inhibition activity, murine anti-human CD39 mAb638 was selected for humanization.

### Example 2.1: Humanization Design of mAb638

The mAb638 variable region genes were employed to create a humanized mAb. In the first step of this process, the amino acid sequences of the VH and VK domains of mAb638 were compared against the available database of human Ig V-gene sequences in order to find the overall best-matching human germline Ig V-gene sequences. Additionally, the framework 4 sequence of VH or VL was compared against the J-region database to find the human framework having the highest homology to the murine VH and VL regions, respectively. For the light chain, the closest human V-gene match was the L6 gene, and for the heavy chain the closest human match was the VH1-2 gene. Humanized variable domain sequences were then designed where the CDR-L1, CDR-L2, and CDR-L3 of the mAb638 light chain variable domain were grafted onto framework sequences of the L6 gene with JK2 framework 4 sequence after CDR-L3; and the CDR-H1, CDR-H2, and CDR-H3 sequences of the mAb638 heavy chain variable domain were grafted onto framework sequences of the VH1-2 with JH6 framework 4 sequence after CDR-H3. A 3-dimensional Fv model of mAb638 was then generated to determine if there were any framework positions where mouse amino acids were critical to support loop structures or the VH/VL interface. Such residues in humanized sequences should be back-mutated to mouse residues at the same position to retain affinity/activity. In the case of the light chain, an Ile to Asn back mutation at position 2 (12N by Kabat numbering), a Tyr to Phe back mutation at position 35 (Y36F by Kabat numbering), an Ala to Ser back mutation at position 42 (A43S by Kabat numbering), a Leu to Val back mutation at position 45 (L46V by Kabat numbering), an Ile to Val back mutation at position 57 (I58V by Kabat numbering), and a Phe to Tyr back mutation at position 70 (F71Y by Kabat numbering) were identified. In the case of the heavy chain, a Met to Ile back mutation at position 48 (M48I by Kabat numbering), an Arg to Lys back mutation at position 67 (R66K by Kabat numbering), a Met to Leu back mutation at position 70 (M69L by Kabat numbering), an Arg to Ala back mutation at position 72 (R71A by Kabat numbering), and a Thr to Lys back mutation at position 74 (T73K by Kabat numbering), were identified as desirable back mutations. Mutated variable domains containing one or more of these back mutations were constructed. See Table 4 below. (Back mutated framework amino acid residues are indicated with double underscore; murine CDRs from the original parental antibody are underlined.)

TABLE 4
Humanized VH/VL design of mAb638

| | SEQ ID | Amino acid sequences |
|---|---|---|
| Humanized VH/VL | NO. | 1234567890123456789012345678901234567890 |
| mAb638-VH.1A | 9 | EVQLVQSGAEVKKPGASVKVSCKASGYTFTSFWMHWVRQA |
| | | PGQGLEWMGNINPRNGATKYNEKFRSRVTMTADTSISTAY |
| | | MELSRLRSDDTAVYYCAREDYDEIYYAMDSWGQGTTVTVS |
| | | S |
| mAb638-VH.1B | 10 | EVQLVQSGAEVKKPGASVKVSCKASGYTFTSFWMHWVRQA |
| | | PGQGLEWMGNINPRNGATKYNEKFRSRVTMTADKSISTAY |
| | | MELSRLRSDDTAVYYCAREDYDEIYYAMDSWGQGTTVTVS |
| | | S |
| mAb638-VH.1C | 11 | EVQLVQSGAEVKKPGASVKVSCKASGYTFTSFWMHWVRQA |
| | | PGQGLEWMGNINPRNGATKYNEKFRSRVTLTADKSISTAY |
| | | MELSRLRSDDTAVYYCAREDYDEIYYAMDSWGQGTTVTVS |
| | | S |
| mAb638-VH.1D | 12 | EVQLVQSGAEVKKPGASVKVSCKASGYTFTSFWMHWVRQA |
| | | PGQGLEWIGNINPRNGATKYNEKFRSRVTLTADKSISTAY |
| | | MELSRLRSDDTAVYYCAREDYDEIYYAMDSWGQGTTVTVS |
| | | S |
| mAb638-VH.1E | 13 | EVQLVQSGAEVKKPGASVKVSCKASGYTFTSFWMHWVRQA |
| | | PGQGLEWIGNINPRNGATKYNEKFRSKVTLTADKSISTAY |
| | | MELSRLRSDDTAVYYCAREDYDEIYYAMDSWGQGTTVTVS |
| | | S |
| mAb638-VK.1A | 14 | ENVLTQSPATLSLSPGERATLSCSASSSVIYMYWYQQKPG |
| | | QAPRLLIYDTSNLASGIPARFSGSGSGTDYTLTISSLEPE |
| | | DFAVYYCQQWSTYPLTFGQGTKLEIK |
| mAb638-VK.1B | 15 | ENVLTQSPATLSLSPGERATLSCSASSSVIYMYWFQQKPG |
| | | QAPRLLIYDTSNLASGIPARFSGSGSGTDYTLTISSLEPE |
| | | DFAVYYCQQWSTYPLTFGQGTKLEIK |
| mAb638-VK.1C | 16 | ENVLTQSPATLSLSPGERATLSCSASSSVIYMYWFQQKPG |
| | | QSPRVLIYDTSNLASGIPARFSGSGSGTDYTLTISSLEPE |
| | | DFAVYYCQQWSTYPLTFGQGTKLEIK |
| mAb638-VK.1D | 17 | ENVLTQSPATLSLSPGERATLSCSASSSVIYMYWFQQKPG |
| | | QSPRVLIYDTSNLASGVPARFSGSGSGTDYTLTISSLEPE |
| | | DFAVYYCQQWSTYPLTFGQGTKLEIK |

The humanized VH and VK genes were produced synthetically and then respectively cloned into vectors containing the human IgG1 and human kappa constant domains. The constant region sequences used are set forth in Table 5, below.

TABLE 5
Human Constant Region Sequence Used in Antibody Humanization

| | SEQ ID | Amino Acid Sequences |
|---|---|---|
| Constant Region | NO. | 1234567890123456789012345678901234567890 |
| human IgG1 | 18 | ASTKGPSVFPLAPSSKSTSGGTAALGCLVKDYFPEPVTVS |
| heavy chain | | WNSGALTSGVHTFPAVLQSSGLYSLSSVVTVPSSSLGTQT |
| constant regions | | YICNVNHKPSNTKVDKKVEPKSCDKTHTCPPCPAPEAAGG |
| (CH1-hinge-CH2- | | PSVFLFPPKPKDTLMISRTPEVTCVVVDVSHEDPEVKFNW |
| CH3) | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| human kappa | 19 | RTVAAPSVFIFPPSDEQLKSGTASVVCLLNNFYPREAKVQ |
| light chain | | WKVDNALQSGNSQESVTEQDSKDSTYSLSSTLTLSKADYE |
| constant region | | KHKVYACEVTHQGLSSPVTKSFNRGEC |

The pairing of the human VH and human VK domains in Table 4 created 20 humanized antibodies, named HuEM0004-38-1 to HuEM0004-38-20 (Table 6). A chimeric antibody with parental mouse VH/VL and human constant sequences was also produced as a positive control, for affinity comparison. There is a possible hydrolytic degradation site AsnGly in the heavy chain CDR2 (CDR-H2) of mAb638, therefore separate mutations of the parental heavy chain to substitute Asn with Gln (N→Q) or Gly with Ala (G→A) at VH positions 55 or 56 (N54Q, G55A substitutions by Kabat numbering) were carried out. See, SEQ ID NO:21 and SEQ ID NO:27. Pairing of the mutated VH with mAb638 VK (SEQ ID NO:8) generated antibodies designated EM0004-38c1 and EM0004-38c2. All recombinant mAbs were expressed and purified.

TABLE 6
Production List of anti-CD39 Humanized
and Chimeric mAb638 Antibodies

| Antibody | VH Region in | VL Region in |
|---|---|---|
| Identifier | Heavy Chain | Light κ Chain |
| HuEM0004-38-1 | mAb638-VH.1A | mAb638-VK.1A |
| HuEM0004-38-2 | mAb638-VH.1B | mAb638-VK.1A |
| HuEM0004-38-3 | mAb638-VH.1C | mAb638-VK.1A |
| HuEM0004-38-4 | mAb638-VH.1D | mAb638-VK.1A |
| HuEM0004-38-5 | mAb638-VH.1E | mAb638-VK.1A |
| HuEM0004-38-6 | mAb638-VH.1A | mAb638-VK.1B |
| HuEM0004-38-7 | mAb638-VH.1B | mAb638-VK.1B |
| HuEM0004-38-8 | mAb638-VH.1C | mAb638-VK.1B |
| HuEM0004-38-9 | mAb638-VH.1D | mAb638-VK.1B |
| HuEM0004-38-10 | mAb638-VH.1E | mAb638-VK.1B |
| HuEM0004-38-11 | mAb638-VH.1A | mAb638-VK.1C |
| HuEM0004-38-12 | mAb638-VH.1B | mAb638-VK.1C |
| HuEM0004-38-13 | mAb638-VH.1C | mAb638-VK.1C |
| HuEM0004-38-14 | mAb638-VH.1D | mAb638-VK.1C |
| HuEM0004-38-15 | mAb638-VH.1E | mAb638-VK.1C |
| HuEM0004-38-16 | mAb638-VH.1A | mAb638-VK.1D |
| HuEM0004-38-17 | mAb638-VH.1B | mAb638-VK.1D |
| HuEM0004-38-18 | mAb638-VH.1C | mAb638-VK.1D |
| HuEM0004-38-19 | mAb638-VH.1D | mAb638-VK.1D |
| HuEM0004-38-20 | mAb638-VH.1E | mAb638-VK.1D |
| EM0004-38c | mAb638 VH | mAb638 VK |
| EM0004-38c1 | mAb638 VH(N54Q) | mAb638 VK |
| EM0004-38c2 | mAb638 VH(G55A) | mAb638 VK |

### Example 2.2: Specificity and ATPase Inhibition Activity of Anti-CD39 Antibodies

Humanized antibodies HuEM04-38-6 to HuEM0004-38-20 and chimeric antibodies EM04-38c, EM004-38c1, and EM0004-38c2 were tested for CD39 specific binding by ELISA as described in Example 1.2 and for inhibition of protein-based ATPase activity as described in Example 1.3. The results are summarized in Table 7.

TABLE 7
Characterization of mAb638 Humanized and Chimeric Antibodies

| | | |
|---|---|---|
| Antibody | ELISA EC50 | ATPase inhibition |

| Identifier | (μg/ml) | IC50 (nM) | inhibition % |
|---|---|---|---|
| HuEM0004-38-6 | — | 139.9 | 64.1 |
| HuEM0004-38-7 | 0.149 | 83.5 | 71.4 |
| HuEM0004-38-8 | 0.097 | 81.8 | 67.9 |
| HuEM0004-38-9 | — | 79.6 | 61.6 |
| HuEM0004-38-10 | 0.175 | 83.2 | 71.5 |
| HuEM0004-38-11 | 0.021 | 65.3 | 99.6 |
| HuEM0004-38-12 | 0.023 | 46.9 | 103.4* |
| HuEM0004-38-13 | 0.023 | 36.2 | 98.7 |
| HuEM0004-38-14 | 0.021 | 36.9 | 98.9 |
| HuEM0004-38-15 | 0.031 | 40.9 | 97.6 |
| HuEM0004-38-16 | 0.027 | 41 | 102.3 |
| HuEM0004-38-17 | 0.044 | 20.4 | 103.3 |
| HuEM0004-38-18 | 0.019 | 18.9 | 98.5 |
| HuEM0004-38-19 | 0.02 | 33.2 | 99.2 |
| HuEM0004-38-20 | 0.031 | 27.3 | 100.4 |
| EM0004-38c1 | 0.023 | 15.6 | 101.5 |
| EM0004-38c2 | 0.016 | 14.3 | 102.9 |
| EM0004-38c | 0.014 | 24.2 | 106.8 |

*protein-based assay experimental data as processed by Graphpad software permitted values exceeding 100% inhibition, which are interpreted as 100% inhibition (complete blocking) of ATPase activity.

### Example 2.3: Cell Surface Binding and Inhibition Activity for Additionally Modified Humanized Anti-CD39 Antibodies

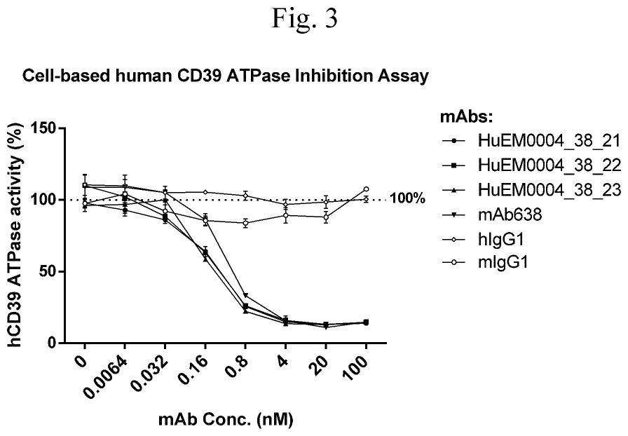

EM0004-38c2, with the G55A mutation, showed a little better binding activity than EM0004-38c1, with N54Q mutation. Compared with other humanized antibodies, HuEM0004-38-17, -18 and -19 showed good binding activity and ATPase inhibition activity. Accordingly, a G55A mutation was introduced in the CDR-H2 in HuEM0004-38-17, -18, and -19 to generate HuEM0004-38-21 (SEQ ID NO:32 (VH) and SEQ ID NO:17 (VK)), HuEM0004-38-22 (SEQ ID NO:33 (VH) and SEQ ID NO:17 (VK)), and HuEM0004-38-23 (SEQ ID NO:34 (VH) and SEQ ID NO:17 (VK)). The three humanized anti-CD39 antibodies were characterized by FACS binding assay and for cell-based inhibition of ATPase activity. The results are set forth in Table 8 and FIG. 3.

TABLE 8
Characterization of mAb638 Humanized
Anti-CD39 Antibodies by FACS

FACS
EC50 (nM)

| Antibody | CHO-K1- | | CHO-K1- |
|---|---|---|---|
| Identifier | human CD39 | SK-MEL-28 | cyno CD39 |
| HuEM0004-38-21 | 6.341 | 4.749 | 6.139 |
| HuEM0004-38-22 | 7.21 | 4.707 | 7.422 |
| HuEM0004-38-23 | 8.36 | 5.474 | 7.177 |

HuEM0004-38-21 had minimal back-mutation(s) while best maintaining the affinity and potency of chimeric mAbs having parental VH and VL domains.

### Example 3: Functional Characterization of Anti-Human CD39 Antibodies

### Example 3.1: Human CD4+ T Cell Proliferation Suppression Assay

To examine the functional activity of the anti-CD39 antibodies of the invention, selected antibodies were used in a human T cell proliferation suppression assay. Human CD4+ cells isolated from fresh PBMC were labeled with carboxyfluorescein succinimidyl ester (CFSE) cell-permeable fluorescent cell staining dye (Sigma, Cat. No. 87444-5MG-F) and mixed with CD2/CD3/CD28 T cell activation beads (Miltenyi Biotec, Cat. No. 130-091-441) according to manufacturer's instructions. The CD4+ T cells were seeded at 1×10⁶ cells/ml, 100 μl/well, onto an assay plate and incubated with 50 μl/well of serially diluted anti-human CD39 antibodies at 37° C. in a 5% CO₂ incubator for 30 minutes. ATP solution at 2 mM was added to the assay plate at 50 μl/well, and the assay plate was incubated at 37° C. in a 5% CO₂ incubator for 3 days, at which point supplemental anti-human CD39 antibodies were added again (50 μl/well). On day 5, supernatant was collected for cytokine analysis and cells were washed twice with 2% FBS in PBS. The CSFE signal was measured on a FACS machine (BD Biosciences FACSCanto II).

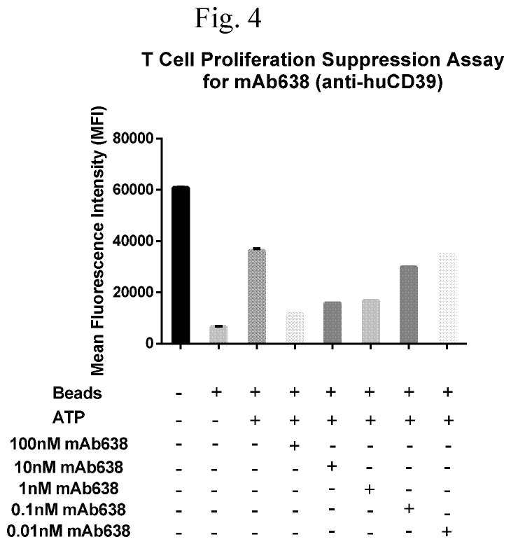

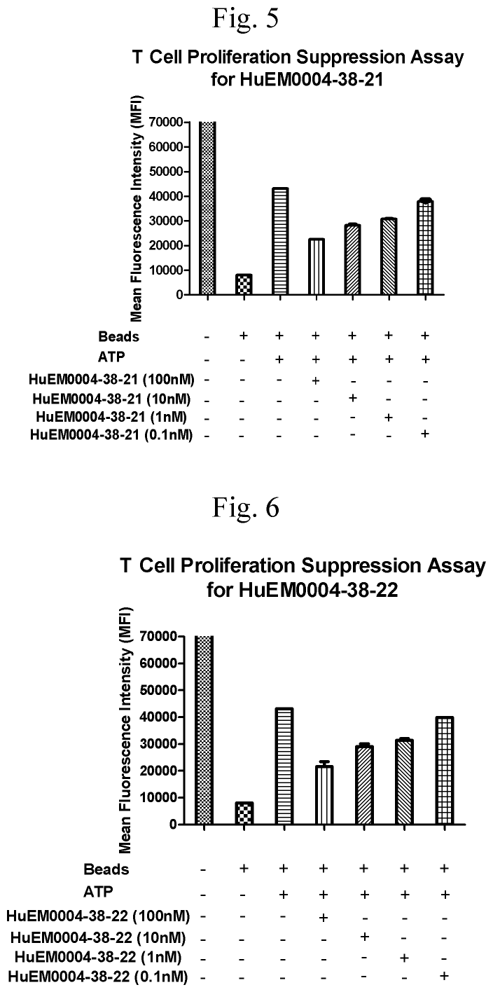

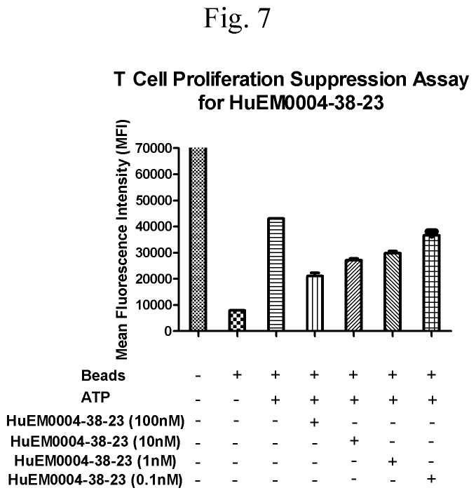

FIG. 4 shows the effect of anti-human CD39 mAb638 in the human CD4+ T cell proliferation suppression assay. The ATPase activity of activated T cells is suppressed by anti-CD39 antibody mAb638 in a concentration-dependent manner. FIGS. 5, 6, and 7 show the effect of humanized anti-human CD39 antibodies HuEM0004-38-21, HuEM0004-38-22, and HuEM0004-38-23 in the human CD4+ T cell proliferation suppression assay. The results show that all the tested humanized antibodies maintain the ATPase inhibition activity of parental antibody mAb638.

### Example 3.2: Affinity Measurement by Surface Plasmon Resonance (SPR)

The binding kinetics of purified antibodies was determined by surface plasmon resonance-based measurements using a Biacore T200 instrument (GE Healthcare). Briefly, goat anti-mouse IgG Fc polyclonal antibody (Genway) was directly immobilized across a biosensor chip, and antibody samples were injected over reaction matrices at a flow rate of 5 μl/min. The association and dissociation rate constants, kₒₙ (M⁻¹s⁻¹) and k_{off}(s⁻¹), respectively, were determined by making kinetic binding measurements of anti-CD39 antibody capture at five different concentrations of a recombinant human or mouse CD39 target protein (huCD39-ECD-His or muCD39-ECD-His). The equilibrium dissociation constant K_{D} (M) of the reaction between antibodies and related target proteins was then calculated from the kinetic rate constants using the formula: K_{D}=k_{off}/kₒₙ. Binding affinities for murine anti-human CD39 antibodies mAb635 and mAb638 are shown in Table 9; binding affinity for rat anti-mouse CD39 antibody mAb605 is shown in Table 10.

TABLE 9
Binding Affinity of Anti-Human CD39 Antibodies by SPR

| | antigen | Kₒₙ | K_{off} | K_{D} |
|---|---|---|---|---|
| mAb ID | target | (M⁻¹) | (s⁻¹) | (M) |
| mAb635 | huCD39- | 1.48 × 10⁵ | 1.06 × 10⁻⁴ | 7.19 × 10⁻¹⁰ |
| | ECD-His | | | |
| mAb638 | huCD39- | 5.74 × 10⁵ | 1.17 × 10⁻⁴ | 2.03 × 10⁻¹⁰ |
| | ECD-His | | | |

HuCD39-ECD-His is a C-terminally hexahistidine-tagged human CD39 extracellular domain (ECD). The ECD sequence is amino acids 38-478 of SEQ ID NO:35.

TABLE 10
Binding Affinity of Anti-Mouse CD39 Antibody mAb605 by SPR

| | antigen | Kₒₙ | K_{off} | K_{D} |
|---|---|---|---|---|
| mAb ID | target | (M⁻¹) | (s⁻¹) | (M) |
| mAb605 | muCD39- | 2.18 × 10⁵ | 1.09 × 10⁻⁴ | 3.89 × 10⁻¹⁰ |
| | ECD-His | | | |

MuCD39-ECD-His is a C-terminally hexahistidine-tagged murine CD39 extracellular domain (ECD). The murine ECD amino acid sequence is set forth below:

(SEQ ID NO: 36)
TQNKPLPENVKYGIVLDAGSSHTNLYIYKWPAEKENDTGVVQQLEECQVK
GPGISKYAQKTDEIGAYLAECMELSTELIPTSKHHQTPVYLGATAGMRLL
RMESEQSADEVLAAVSTSLKGYPFDFQGAKIITGQEEGAYGWITINYLLG
RFTQEQSWLSLISDSQKQETFGALDLGGASTQITFVPQNSTIESPENSLQ
FRLYGEDYTVYTHSFLCYGKDQALWQKLAKDIQVSSGGVLKDPCFNPGYE
KVVNVSELYGTPCTERFEKKLPFDQFRIQGTGDYEQCHQSILELFNNSHC
PYSQCAFNGVFLPPLHGSFGAFSAFYFVMDFFKKVAKNSVISQEKMTEIT
KNECSKSWEETKTSYPSVKEKYLSEYCFSGAYILSLLQGYNFTDSSWEQI
HFMGKIKDSNAGWTLGYMLNLTNMIPAEQPLSPPLPHSTY

The foregoing data indicate that the anti-human CD39 and anti-mouse CD39 antibodies tested showed high affinity for the target antigen.

### Example 3.3: Affinity of Humanized Anti-HuCD39 Antibodies by Octet® RED

Anti-CD39 antibodies were characterized for affinities and binding kinetics using the Octet®RED96 biolayer interferometry system (Pall FortéBio LLC). Purified anti-CD39 antibodies were captured by Anti-Mouse IgG Fc Capture (AMC) biosensors or Anti-Human IgG Fc Capture (AHC) biosensors at a concentration of 100 nM for 30 seconds. Biosensors were then dipped into running buffer (1X pH7.2 PBS, 0.05% Tween 20, 0.1% BSA) for 60 seconds to check baseline. Binding was measured by dipping sensors into purified recombinant human CD39 protein, 3-fold serial diluted from 200 nM, for 120 seconds. Dissociation was followed by dipping sensors into running buffer for 600 seconds. The association and dissociation curves were fitted to a 1:1 Langmuir binding model using Fortebio Data Analysis software (Pall FortéBio LLC). Affinity determinations are shown in Table 11, below. The results show that the humanized antibodies retained the binding affinity for CD39 antigen target exhibited by the parental murine antibody.

TABLE 11
Affinity of Humanized Antibodies to Human
CD39 by Octet ® RED

| | antigen | Kₒₙ | K_{off} | K_{D} |
|---|---|---|---|---|
| mAb ID | target | (M⁻¹) | (s⁻¹) | (M) |
| EM0004- | huCD39- | 1.39 × 10⁵ | 3.16 × 10⁻⁴ | 2.28 × 10⁻⁹ |
| 38-21 | ECD-His | | | |
| EM0004- | huCD39- | 1.38 × 10⁵ | 3.48 × 10⁻⁴ | 2.53 × 10⁻⁹ |
| 38-22 | ECD-His | | | |
| EM0004- | huCD39- | 1.43 × 10⁵ | 3.65 × 10⁻⁴ | 2.55 × 10⁻⁹ |
| 38-23 | ECD-His | | | |
| mAb038 | huCD39- | 1.88 × 10⁵ | 4.26 × 10⁻⁴ | 2.27 × 10⁻⁹ |
| | ECD-His | | | |

The contents of all publications (including literature references, patents, patent applications, and websites) cited throughout this application are hereby expressly incorporated by reference in their entirety for any purpose, as are the references cited therein. The practice of the present embodiments will employ, unless otherwise indicated, conventional techniques of immunology, molecular biology and cell biology, which are well known in the art. The embodiments may be carried out in other specific forms without departing from the spirit or essential characteristics thereof. The foregoing embodiments are therefore to be considered in all respects illustrative rather than limiting. The invention is defined as indicated by the appended claims, and all changes that come within the meaning and range of equivalency of the claims are therefore intended to be embraced herein.

What is claimed is:

1. An antibody, or an antigen-binding portion thereof, capable of binding human CD39, wherein the antigen-binding portion of the antibody comprises a set of six CDRs, CDR-H1, CDR-H2, CDR-H3, CDR-L1, CDR-L2, and CDR-L3, selected from the group of CDR sets as follows:

| CDR | | CDR | |
|---|---|---|---|
| Set No. | CDR | Amino Acid Sequence | SEQ ID NO: |
| 1 | CDR-H1 | DNYLA | residues 31-35 of SEQ ID NO: 1 |
| | CDR-H2 | ISFEGISTYYGDSVK | residues 51-65 of SEQ ID NO: 1 |
| | CDR-H3 | RSYGSRAMDA | residues 99-108 of SEQ ID NO: 1 |
| | CDR-L1 | RSSQSLVHSNGNTYLS | residues 24-39 of SEQ ID NO: 2 |
| | CDR-L2 | VSSRF | residues 56-70 of SEQ ID NO: 2 |
| | CDR-L3 | LQTTQYPY | residues 94-101 of SEQ ID NO: 2 |
| 2 | CDR-H1 | RFGIH | residues 31-35 of SEQ ID NO: 3 |
| | CDR-H2 | VIWGGGSTDYDAGFIS | residues 50-65 of SEQ ID NO: 3 |
| | CDR-H3 | KDDDYLEAWFAH | residues 98-109 of SEQ ID NO: 3 |
| | CDR-L1 | KASENVGTYVS | residues 24-34 of SEQ ID NO: 4 |
| | CDR-L2 | GASSRYT | residues 50-56 of SEQ ID NO: 4 |
| | CDR-L3 | GQSYSYPYT | residues 89-97 of SEQ ID NO: 4 |
| 3 | CDR-H1 | SYWMH | residues 31-35 of SEQ ID NO: 5 |
| | CDR-H2 | EIDPSDDYTNYNQNFKG | residues 50-66 of SEQ ID NO: 5 |
| | CDR-H3 | EENGIYYYNKGYLDV | residues 99-113 of SEQ ID NO: 5 |
| | CDR-L1 | SSKSLLHSNGITYLY | residues 25-39 of SEQ ID NO: 6 |
| | CDR-L2 | QMSNLAS | residues 55-61 of SEQ ID NO: 6 |
| | CDR-L3 | AQNLELPYT | residues 94-102 of SEQ ID NO: 6 |
| 4 | CDR-H1 | SFWMH | residues 31-35 of SEQ ID NO: 7 |
| | CDR-H2 | NINPRNGATKYNEKFRS | residues 50-66 of SEQ ID NO: 7 |
| | CDR-H3 | EDYDEIYYAMDS | residues 99-110 of SEQ ID NO: 7 |
| | CDR-L1 | SASSSVIYMY | residues 24-33 of SEQ ID NO: 8 |
| | CDR-L2 | DTSNLAS | residues 49-55 of SEQ ID NO: 8 |
| | CDR-L3 | QQWSTYPLT | residues 88-96 of SEQ ID NO: 8 |
| 5 | CDR-H1 | SFWMH | SEQ ID NO: 20 |
| | CDR-H2 | NINPRQGATKYNEKFRS | SEQ ID NO: 21 |
| | CDR-H3 | EDYDEIYYAMDS | SEQ ID NO: 22 |
| | CDR-L1 | SASSSVIYMY | SEQ ID NO: 23 |
| | CDR-L2 | DTSNLAS | SEQ ID NO: 24 |
| | CDR-L3 | QQWSTYPLT | SEQ ID NO: 25 |
| 6 | CDR-H1 | SFWMH | SEQ ID NO: 26 |
| | CDR-H2 | NINPRNAATKYNEKFRS | SEQ ID NO: 27 |
| | CDR-H3 | EDYDEIYYAMDS | SEQ ID NO: 28 |
| | CDR-L1 | SASSSVIYMY | SEQ ID NO: 29 |
| | CDR-L2 | DTSNLAS | SEQ ID NO: 30 |
| | CDR-L3 | QQWSTYPLT | SEQ ID NO: 31 |

2. An anti-CD39 antibody comprising VH and VL domains, wherein the two variable domains comprise amino acid sequences selected from the group consisting of:

| | |
|---|---|
| SEQ ID NO: 1 and SEQ ID NO: 2 | SEQ ID NO: 11 and SEQ ID NO: 14 |
| SEQ ID NO: 3 and SEQ ID NO: 4 | SEQ ID NO: 11 and SEQ ID NO: 15 |
| SEQ ID NO: 5 and SEQ ID NO: 6 | SEQ ID NO: 11 and SEQ ID NO: 16 |
| SEQ ID NO: 7 and SEQ ID NO: 8 | SEQ ID NO: 11 and SEQ ID NO: 17 |
| SEQ ID NO: 9 and SEQ ID NO: 14 | SEQ ID NO: 12 and SEQ ID NO: 14 |
| SEQ ID NO: 9 and SEQ ID NO: 15 | SEQ ID NO: 12 and SEQ ID NO: 15 |
| SEQ ID NO: 9 and SEQ ID NO: 16 | SEQ ID NO: 12 and SEQ ID NO: 16 |
| SEQ ID NO: 9 and SEQ ID NO: 17 | SEQ ID NO: 12 and SEQ ID NO: 17 |
| SEQ ID NO: 10 and SEQ ID NO: 14 | SEQ ID NO: 13 and SEQ ID NO: 14 |
| SEQ ID NO: 10 and SEQ ID NO: 15 | SEQ ID NO: 13 and SEQ ID NO: 15 |
| SEQ ID NO: 10 and SEQ ID NO: 16 | SEQ ID NO: 13 and SEQ ID NO. 16 |
| SEQ ID NO: 10 and SEQ ID NO: 17 | SEQ ID NO: 13 and SEQ ID NO: 17 |
| SEQ ID NO: 32 and SEQ ID NO: 17 | SEQ ID NO: 33 and SEQ ID NO: 17 |

and SEQ ID NO:34 and SEQ ID NO: 17.

3. The antibody or antigen-binding fragment thereof according to claim 1, further comprising an Fe region comprising the sequence of amino acids of residues 104-330 of SEQ ID NO: 18.

4. A nucleic acid molecule encoding a monoclonal antibody binding to human CD39, wherein the nucleotide sequence encoding the heavy chain comprises nucleotides encoding CDR-H1, CDR-H2, and CDR-H3 selected from heavy chain CDR sets 1-6 as follows

| CDR | | CDR | |
|---|---|---|---|
| Set No. | CDR | Amino Acid Sequence | SEQ ID NO: |
| 1 | CDR-H1 | DNYLA | residues 31-35 of SEQ ID NO: 1 |
| | CDR-H2 | ISFEGISTYYGDSVK | residues 51-65 of SEQ ID NO: 1 |
| | CDR-H3 | RSYGSRAMDA | residues 99-108 of SEQ ID NO: 1 |
| 2 | CDR-H1 | RFGIH | residues 31-35 of SEQ ID NO: 3 |
| | CDR-H2 | VIWGGGSTDYDAGFIS | residues 50-65 of SEQ ID NO: 3 |
| | CDR-H3 | KDDDYLEAWFAH | residues 98-109 of SEQ ID NO: 3 |
| 3 | CDR-H1 | SYWMH | residues 31-35 of SEQ ID NO: 5 |
| | CDR-H2 | EIDPSDDYTNYNQNFKG | residues 50-66 of SEQ ID NO: 5 |
| | CDR-H3 | EENGIYYYNKGYLDV | residues 99-113 of SEQ ID NO: 5 |
| 4 | CDR-H1 | SFWMH | residues 31-35 of SEQ ID NO: 7 |
| | CDR-H2 | NINPRNGATKYNEKFRS | residues 50-66 of SEQ ID NO: 7 |
| | CDR-H3 | EDYDEIYYAMDS | residues 99-110 of SEQ ID NO: 7 |
| 5 | CDR-H1 | SFWMH | SEQ ID NO: 20 |
| | CDR-H2 | NINPRQGATKYNEKFRS | SEQ ID NO: 21 |
| | CDR-H3 | EDYDEIYYAMDS | SEQ ID NO: 22 |
| 6 | CDR-H1 | SFWMH | SEQ ID NO: 26 |
| | CDR-H2 | NINPRNAATKYNEKFRS | SEQ ID NO: 27 |
| | CDR-H3 | EDYDEIYYAMDS | SEQ ID NO: 28 |

and wherein the nucleotide sequence encoding the light chain comprises nucleotides encoding CDR-L1, CDR-L2, and CDR-L3 selected from light chain CDR sets 1-6 as follows

| CDR | | CDR | |
|---|---|---|---|
| Set No. | CDR | Amino Acid Sequence | SEQ ID NO: |
| 1 | CDR-L1 | RSSQSLVHSNGNTYLS | residues 24-39 of SEQ ID NO: 2 |
| | CDR-L2 | VSSRF | residues 56-70 of SEQ ID NO: 2 |
| | CDR-L3 | LQTTQYPY | residues 94-101 of SEQ ID NO: 2 |
| 2 | CDR-L1 | KASENVGTYVS | residues 24-34 of SEQ ID NO: 4 |
| | CDR-L2 | GASSRYT | residues 50-56 of SEQ ID NO: 4 |
| | CDR-L3 | GQSYSYPYT | residues 89-97 of SEQ ID NO: 4 |
| 3 | CDR-L1 | SSKSLLHSNGITYLY | residues 25-39 of SEQ ID NO: 6 |
| | CDR-L2 | QMSNLAS | residues 55-61 of SEQ ID NO: 6 |
| | CDR-L3 | AQNLELPYT | residues 94-102 of SEQ ID NO: 6 |
| 4 | CDR-L1 | SASSSVIYMY | residues 24-33 of SEQ ID NO: 8 |
| | CDR-L2 | DTSNLAS | residues 49-55 of SEQ ID NO: 8 |
| | CDR-L3 | QQWSTYPLT | residues 88-96 of SEQ ID NO: 8 |
| 5 | CDR-L1 | SASSSVIYMY | SEQ ID NO: 23 |
| | CDR-L2 | DTSNLAS | SEQ ID NO: 24 |
| | CDR-L3 | QQWSTYPLT | SEQ ID NO: 25 |
| 6 | CDR-L1 | SASSSVIYMY | SEQ ID NO: 29 |
| | CDR-L2 | DTSNLAS | SEQ ID NO: 30 |
| | CDR-L3 | QQWSTYPLT | SEQ ID NO: 31. |

5. An expression vector comprising a nucleic acid molecule encoding a monoclonal antibody binding to human CD39 according to claim 1.

6. A host cell comprising an expression vector according to claim 5.

7. A method for producing a monoclonal antibody binding to CD39 according to claim 1, wherein the method comprises the following steps:
(a) culturing the host cell according to claim 6 under expression conditions to express a monoclonal antibody binding to human CD39; and
(b) isolating and purifying the monoclonal antibody binding to human CD39 obtained in step (a).

8. A pharmaceutical composition comprising at least one anti-CD39 antibody or antigen-binding fragment thereof according to any one of claims 1-3, and a pharmaceutically acceptable carrier.

9. A method of treating a disorder wherein CD39-mediated activity is detrimental, comprising administering to a subject in need thereof an effective amount of a pharmaceutical composition according to claim 8.

10. The method according to claim 9, wherein said disorder is a cancer selected from melanoma (e.g., metastatic malignant melanoma), renal cancer, pancreatic adenocarcinoma, breast cancer, colon cancer, lung cancer (e.g. non-small cell lung cancer), head and neck, liver cancer, ovarian cancer, bladder cancer, kidney cancer, salivary cancer, stomach cancer, gliomas cancer, thyroid cancer, thymic cancer, epithelial cancer, gastric cancer, and lymphoma.

11. Use of an antibody or antigen-binding fragment thereof according to any one of claims 1-3 in the preparation of a medicament for treating a disease or disorder in which CD39-mediated activity is detrimental.
